SEQUENCE LISTING (213 sequences)

SEQ ID NO: 1 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 2 - protein, 148 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 3 - protein, 17 aa, Homo sapiens
LGWLCLLLLPIPLIVWV

SEQ ID NO: 4 - protein, 144 aa, Homo sapiens
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITDSENSNFRNEIQSLV

SEQ ID NO: 5 - protein, 122 aa, Artificial Sequence
  VH domain from anti-Fas antibody
QVQLQQWGAGLLKPSETLSLICAVYGGSFSTYYWTWIRQPPGKGLEWIGEINHRGTTNYSPSLKSRVTISVDTSKNHISLNLTSVTAADTALYYCARGLLWIGEGDYGLDVWGQGTTVTVSS

SEQ ID NO: 6 - protein, 108 aa, Artificial Sequence
  VL domain from anti-Fas antibody
DIQMTQSPSSLSASVGDRVTITCRASQGIRRWLAWYQQKPEKAPKSLIYAASSLQSGVPSRFSGSGSGTDFTLTISSLQPEDFATYYCQQYNSYPYTFGQGTKLEIKR

SEQ ID NO: 7 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 8 - protein, 189 aa, Homo sapiens
ALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 9 - protein, 4 aa, Artificial Sequence
  ER retention signal, C-terminal signal
KDEL

SEQ ID NO: 10 - protein, 4 aa, Artificial Sequence
  ER retention signal, C-terminal signal
  misc_feature (3)..(4): Xaa can be any naturally occurring amino acid
KKXX

SEQ ID NO: 11 - protein, 5 aa, Artificial Sequence
  ER retention signal, C-terminal signal
  misc_feature (2)..(2): Xaa can be any naturally occurring amino acid
  misc_feature (4)..(5): Xaa can be any naturally occurring amino acid
KXKXX

SEQ ID NO: 12 - protein, 4 aa, Artificial Sequence
  ER retention signal, RXR(R) motif of potassium channels
  misc_feature (2)..(2): Xaa can be any naturally occurring amino acid
RXRR

SEQ ID NO: 13 - protein, 6 aa, Artificial Sequence
  Golgi retention sequence
SEKDEL

SEQ ID NO: 14 - protein, 15 aa, Artificial Sequence
  Golgi retention sequence, tail of adenoviral E19 protein
KYKSRRSFIDEKKMP

SEQ ID NO: 15 - protein, 10 aa, Artificial Sequence
  Golgi retention sequence, HLA invariant chain
MHRRRSRSCR

SEQ ID NO: 16 - protein, 3 aa, Artificial Sequence
  Golgi retention sequence, HLA invariant chain
  misc_feature (2)..(2): Xaa can be any naturally occurring amino acid
  MISC_FEATURE (3)..(3): Xaa may be Asp or Glu
KXX

SEQ ID NO: 17 - protein, 4 aa, Artificial Sequence
  Golgi retention sequence, HLA invariant chain
YQRL

SEQ ID NO: 18 - protein, 11 aa, Artificial Sequence
  KDEL sequence in Fas-retaining molecule
DPAEPSEKDEL

SEQ ID NO: 19 - protein, 6 aa, Artificial Sequence
  retention signal, tail of adenoviral E19 protein sequence
DEKKMP

SEQ ID NO: 20 - protein, 7 aa, Artificial Sequence
  intracellular retention signal, TYRP-1 intracellular domain
NQPLLTD

SEQ ID NO: 21 - protein, 5 aa, Artificial Sequence
  linker
GGGGS

SEQ ID NO: 22 - protein, 10 aa, Artificial Sequence
  linker
GGGGSGGGGS

SEQ ID NO: 23 - protein, 13 aa, Artificial Sequence
  linker
SGGGSGGGSGGGS

SEQ ID NO: 24 - protein, 19 aa, Artificial Sequence
  anti-Fas KDEL molecule with an Ig Kappa leader sequence
MGWSCIILFLVATATGVHS

SEQ ID NO: 25 - protein, 276 aa, Artificial Sequence
  anti-Fas-KDEL sequence
MGWSCIILFLVATATGVHSDIQMTQSPSSLSASVGDRVTITCRASQGIRRWLAWYQQKPEKAPKSLIYAASSLQSGVPSRFSGSGSGTDFTLTISSLQPEDFATYYCQQYNSYPYTFGQGTKLEIKRSGGGGSGGGGSGGGGSQVQLQQWGAGLLKPSETLSLICAVYGGSFSTYYWTWIRQPPGKGLEWIGEINHRGTTNYSPSLKSRVTISVDTSKNHISLNLTSVTAADTALYYCARGLLWIGEGDYGLDVWGQGTTVTVSSDPAEPSEKDEL

SEQ ID NO: 26 - protein, 60 aa, Artificial Sequence
  truncated Fas endodomain
KRKEVQKTCRKHRKENQGSHESPTLNPETVAAEKIQTIILKDITSDSENSNFRNEIQSLV

SEQ ID NO: 27 - protein, 225 aa, Artificial Sequence
  truncated Fas, or FasdeltaDD
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAAEKIQTIILKDITSDSENSNFRNEIQSLV

SEQ ID NO: 28 - protein, 332 aa, Artificial Sequence
  Fas with D260V point mutation
MLGIWTLLPLVLTSVARLSSKSWAQVTDINSKGLELRKTVTTVETQNLEGLHHDGQFCHKPCPPGERKARDCTWGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGWEAKIVEIKNDNVQDTAEQKVQLLRNWHQLHGKKEAYDTLIKDLKKANLCTLAEKIQTIILKDITSDSENSNFRNEIQSLV

SEQ ID NO: 29 - protein, 154 aa, Artificial Sequence
  DcR2 endodomain
RKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 30 - protein, 58 aa, Artificial Sequence
  GITR endodomain
QLGLHIWQLRSQCMWPRETQLLLEVPPSTEDARSCQFPEEERGERSAEEKGRLGDLWV

SEQ ID NO: 31 - protein, 188 aa, Artificial Sequence
  CD30 endodomain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 32 - protein, 138 aa, Artificial Sequence
  XEDAR endodomain
LYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 33 - protein, 62 aa, Artificial Sequence
  CD40 endodomain
KKVAKKPTNKAPHPKQEPQEINFPDDLPGSNTAAPVQETLHGCQPVTQEDGKESRISVQERQ

SEQ ID NO: 34 - protein, 50 aa, Artificial Sequence
  CD27 endodomain
RKQRRKYRSNKGESPVEPAEPCHYSCPREEEGSTIPIQEDYRKPEPACSP

SEQ ID NO: 35 - protein, 107 aa, Artificial Sequence
  BCMA endodomain
RKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 36 - protein, 28 aa, Artificial Sequence
  Fn14 endodomain
RRCRRREKFTTPIEETGGEGCPAVALIQ

SEQ ID NO: 37 - protein, 42 aa, Artificial Sequence
  41BB endodomain
KRGRKKLLYIFKQPFMRPVQTTQEEDGCSCRFPEEEEGGCEL

SEQ ID NO: 38 - DNA, 19 bp, Artificial Sequence
  sequence tested, predicted as siRNA for Fas mRNA
ggacattactagtgactca

SEQ ID NO: 39 - DNA, 19 bp, Artificial Sequence
  sequence tested, predicted as siRNA for Fas mRNA
gttcaactgcttcgtaatt

SEQ ID NO: 40 - DNA, 21 bp, Artificial Sequence
  sense siRNA sequence (knocks down Fas expression)
gugcaagugcaaaccagactt

SEQ ID NO: 41 - DNA, 21 bp, Artificial Sequence
  antisense siRNA sequence (knocks down Fas expression)
gucugguuugcacuugcactt

SEQ ID NO: 42 - DNA, 58 bp, Artificial Sequence
  anti-Fas shRNA sequence
ccgggtgcagatgtaaaccaaacttctcgagaagtttggtttacatctgcactttttg

SEQ ID NO: 43 - DNA, 20 bp, Artificial Sequence
  Fas-gRNA encoding sequence
gagggtccagatgcccagca

SEQ ID NO: 44 - RNA, 20 nt, Artificial Sequence
  Fas-targeting gRNA sequence
ggaguugaugucagucacuu

SEQ ID NO: 45 - RNA, 20 nt, Artificial Sequence
  Fas-targeting gRNA sequence
gugacugacaucaacuccaa

SEQ ID NO: 46 - RNA, 20 nt, Artificial Sequence
  Fas-targeting gRNA sequence
cuuccucaauuccaaucccu

SEQ ID NO: 47 - RNA, 20 nt, Artificial Sequence
  Fas-targeting gRNA sequence
ugacaucaacuccaagggau

SEQ ID NO: 48 - protein, 109 aa, Artificial Sequence
  human CD3zeta endodomain
SRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 41 aa, Artificial Sequence
  CD28 endodomain
RSKRSRLLHSDYMNMTPRRPGPTRKHYQPYAPPRDFAAYRS

SEQ ID NO: 50 - protein, 38 aa, Artificial Sequence
  ICOS endodomain
CWLTKKKYSSSVHDPNGEYMFMRAVNTAKKSRLTDVTL

SEQ ID NO: 51 - protein, 41 aa, Artificial Sequence
  CTLA4 endodomain
AVSLSKMLKKRSPLTTGVYVKMPPTEPECEKQFQPYFIPIN

SEQ ID NO: 52 - protein, 42 aa, Artificial Sequence
  OX-40 endodomain
ALYLLRRDQRLPPDAHKPPGGGSFRTPIQEEQADAHSTLAKI

SEQ ID NO: 53 - protein, 107 aa, Artificial Sequence
  TACI endodomain
KKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 54 - protein, 115 aa, Artificial Sequence
  CD2 endodomain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSS

SEQ ID NO: 55 - protein, 271 aa, Artificial Sequence
  mutant FasL having a deletion of residues 126-135
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 56 - protein, 261 aa, Artificial Sequence
  mutant FasL having a deletion of residues 126-145
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 57 - protein, 271 aa, Homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 58 - protein, 270 aa, Artificial Sequence
  mutant DcR3 sequence
VAETPTYPWRDAETGERLVCAQCPPGTFVQRPCRRDSPTTCGPCPPRHYTQFWNLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLALAARLTELLGAQDGALLVRLLQALRVARMPGLERSVRERFLPVH

SEQ ID NO: 59 - protein, 46 aa, Artificial Sequence
  sequence suitable for tethering DcR3 to the plasma membrane, CD8stk
PTTTPAPRPPTPAPTIASQPLSLRPEACRPAAGGAVHTRGLDFACD

SEQ ID NO: 60 - protein, 15 aa, Artificial Sequence
  sequence suitable for tethering DcR3 to the plasma membrane, Hinge
EPKSCDKTHTCPPCP

SEQ ID NO: 61 - protein, 51 aa, Artificial Sequence
  sequence suitable for tethering DcR3 to the plasma membrane, COMP
AGSDLGPQMLRELQETNAALQDVRELLRQQVREITFLKNTVMECDACGSGK

SEQ ID NO: 62 - protein, 110 aa, Artificial Sequence
  sequence suitable for tethering DcR3 to the plasma membrane, CH1-Hinge
KGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 63 - protein, 327 aa, Artificial Sequence
  sequence suitable for tethering DcR3 to the plasma membrane, CH1-Hinge-CH2-CH3
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 107 aa, Artificial Sequence [sequence omitted]

SEQ ID NO: 65 - protein, 41 aa, Artificial Sequence
  sequence suitable for tethering DcR3 to the plasma membrane, CD28 Stalk
AKIEVMYPPPYLDNEKSNGTIIHVKGKHLCPSPLFPGPSKP

SEQ ID NO: 66 - protein, 4 aa, Artificial Sequence
  polar anchor
RKKR

SEQ ID NO: 67 - protein, 50 aa, Artificial Sequence
  rigid linker with truncations
LEAEAAAKEAAAKEAAAKEAAAKALEAEAAAKEAAAKEAAAKEAAAKALE

SEQ ID NO: 68 - DNA, 20 bp, Artificial Sequence
  gRNA targeting TCR alpha chain constant region (TRAC-gRNA)
agagtctctcagctggtaca

SEQ ID NO: 69 - DNA, 20 bp, Artificial Sequence
  gRNA targeting TCR beta chain constant region (TRBC-gRNA)
gcagtatctggagtcattga

SEQ ID NO: 70 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 71 - protein, 521 aa, Artificial Sequence
  calcineurin A alpha isoform
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 72 - protein, 170 aa, Artificial Sequence
  calcineurin B, type 1
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 73 - protein, 20 aa, Artificial Sequence
  cleavage site, 2A-like sequence
RAEGRGSLLTCGDVEENPGP

SEQ ID NO: 74 - protein, 4 aa, Artificial Sequence
  intracellular retention signal
  misc_feature (4)..(4): Xaa can be any naturally occurring amino acid
NPFX

SEQ ID NO: 75 - protein, 4 aa, Artificial Sequence
  tyrosine-based sorting signal
  misc_feature (3)..(3): Xaa can be any naturally occurring amino acid
NPXY

SEQ ID NO: 76 - protein, 4 aa, Artificial Sequence
  tyrosine-based sorting signal
  misc_feature (2)..(3): Xaa can be any naturally occurring amino acid
  MISC_FEATURE (4)..(4): Xaa is an amino acid with a bulky hydrophobic side chain
YXXX

SEQ ID NO: 77 - protein, 12 aa, Homo sapiens
INFDNPVYQKTT

SEQ ID NO: 78 - protein, 12 aa, Homo sapiens
VEIGNPTYKMYE

SEQ ID NO: 79 - protein, 12 aa, Homo sapiens
TNFTNPVYATLY

SEQ ID NO: 80 - protein, 12 aa, Drosophila sp.
GNFANPVYESMY

SEQ ID NO: 81 - protein, 12 aa, Caenorhabditis elegans
TTFTNPVYELED

SEQ ID NO: 82 - protein, 12 aa, Caenorhabditis elegans
LRVDNPLYDPDS

SEQ ID NO: 83 - protein, 12 aa, Homo sapiens
IIFENPMYSARD

SEQ ID NO: 84 - protein, 12 aa, Homo sapiens
TNFENPIYAQME

SEQ ID NO: 85 - protein, 12 aa, Homo sapiens
DTGENPIYKSAV

SEQ ID NO: 86 - protein, 11 aa, Homo sapiens
TTVVNPKYEGK

SEQ ID NO: 87 - protein, 12 aa, Drosophila sp.
WDTENPIYKQAT

SEQ ID NO: 88 - protein, 11 aa, Drosophila sp.
STFKNPMYAGK

SEQ ID NO: 89 - protein, 12 aa, Homo sapiens
HGYENPTYRFLE

SEQ ID NO: 90 - protein, 12 aa, Homo sapiens
NGYENPTYKFFE

SEQ ID NO: 91 - protein, 12 aa, Drosophila sp.
NGYENPTYKYFE

SEQ ID NO: 92 - protein, 12 aa, Homo sapiens
YASSNPEYLSAS

SEQ ID NO: 93 - protein, 12 aa, Homo sapiens
GSVQNPVYHNQP

SEQ ID NO: 94 - protein, 12 aa, Homo sapiens
TAVGNPEYLNTV

SEQ ID NO: 95 - protein, 12 aa, Homo sapiens
ISLDNPDYQQDF

SEQ ID NO: 96 - protein, 11 aa, Homo sapiens
RKRSHAGYQTI

SEQ ID NO: 97 - protein, 10 aa, Homo sapiens
KHHHAGYEQF

SEQ ID NO: 98 - protein, 11 aa, Artificial Sequence
  chicken LAMP-2a sequence
KKHHNTGYEQF

SEQ ID NO: 99 - protein, 11 aa, Artificial Sequence
  chicken LAMP-2b sequence
RRKSRTGYQSV

SEQ ID NO: 100 - protein, 11 aa, Artificial Sequence
  chicken LAMP-2c sequence
RRKSYAGYQTL

SEQ ID NO: 101 - protein, 12 aa, Drosophila sp.
RRRSTSRGYMSF

SEQ ID NO: 102 - protein, 11 aa, Artificial Sequence
  earthworm LAMP sequence
RKRSRRGYESV

SEQ ID NO: 103 - protein, 10 aa, Homo sapiens
KSIRSGYEVM

SEQ ID NO: 104 - protein, 12 aa, Homo sapiens
HCGGPRPGYETL

SEQ ID NO: 105 - protein, 12 aa, Mus musculus
HCRTRRAEYETL

SEQ ID NO: 106 - protein, 10 aa, Homo sapiens
RRRPSAYQAL

SEQ ID NO: 107 - protein, 9 aa, Homo sapiens
RRRSYQNIP

SEQ ID NO: 108 - protein, 10 aa, Homo sapiens
KKHCSYQDIL

SEQ ID NO: 109 - protein, 10 aa, Mus musculus
RRRSAYQDIR

SEQ ID NO: 110 - protein, 11 aa, Rattus norvegicus
RKRRRSYQDIM

SEQ ID NO: 111 - protein, 13 aa, Rattus norvegicus
KFCKSKERNYHTL

SEQ ID NO: 112 - protein, 14 aa, Drosophila sp.
KFYKARNERNYHTL

SEQ ID NO: 113 - protein, 14 aa, Homo sapiens
KIRLRCQSSGYQRI

SEQ ID NO: 114 - protein, 14 aa, Mus musculus
KIRQRHQSSAYQRI

SEQ ID NO: 115 - protein, 15 aa, Homo sapiens
HFCLYRKRPGYDQLN

SEQ ID NO: 116 - protein, 11 aa, Homo sapiens
SLSRGSGYKEI

SEQ ID NO: 117 - protein, 13 aa, Homo sapiens
RRLRKGYTPLMET

SEQ ID NO: 118 - protein, 14 aa, Homo sapiens
RRAGHSSYTPLPGS

SEQ ID NO: 119 - protein, 18 aa, Dictyostelium sp.
KKLRQQKQQGYQAIINNE

SEQ ID NO: 120 - protein, 15 aa, Dictyostelium sp.
RSKSNQNQSYNLIQL

SEQ ID NO: 121 - protein, 17 aa, Dictyostelium sp.
RKTFYNNNQYNGYNIIN

SEQ ID NO: 122 - protein, 10 aa, Homo sapiens
PLSYTRFSLA

SEQ ID NO: 123 - protein, 11 aa, Homo sapiens
MTKEYQDLQHL

SEQ ID NO: 124 - protein, 10 aa, Homo sapiens
SYKYSKVNKE

SEQ ID NO: 125 - protein, 10 aa, Homo sapiens
PAAYRGVGDD

SEQ ID NO: 126 - protein, 10 aa, Homo sapiens
TGVYVKMPPT

SEQ ID NO: 127 - protein, 10 aa, Homo sapiens
LISYKGLPPE

SEQ ID NO: 128 - protein, 11 aa, Rattus norvegicus
ASDYQRLNLKL

SEQ ID NO: 129 - protein, 11 aa, Human immunodeficiency virus type 1
RQGYSPLSFQT

SEQ ID NO: 130 - protein, 19 aa, Homo sapiens
RMQAQPPGYRHVADGEDHA

SEQ ID NO: 131 - protein, 7 aa, Artificial Sequence
  dileucine-based sorting signal
  MISC_FEATURE (1)..(1): Xaa may be Asp or Glu
  misc_feature (2)..(4): Xaa can be any naturally occurring amino acid
  MISC_FEATURE (7)..(7): Xaa may be Leu or Ile
XXXXLLX

SEQ ID NO: 132 - protein, 9 aa, Homo sapiens
SDKQTLLPN

SEQ ID NO: 133 - protein, 9 aa, Rattus norvegicus
DERAPLIRT

SEQ ID NO: 134 - protein, 9 aa, Homo sapiens
QEKDPLLKN

SEQ ID NO: 135 - protein, 9 aa, Coturnix japonica
TERNPLLKS

SEQ ID NO: 136 - protein, 9 aa, Homo sapiens
GENSPLLSG

SEQ ID NO: 137 - protein, 9 aa, Homo sapiens
EEKQPLLME

SEQ ID NO: 138 - protein, 9 aa, Oryzias latipes
GERQPLLQS

SEQ ID NO: 139 - protein, 9 aa, Artificial Sequence
  chicken tyrosinase dileucine-based sorting signal
PEIQPLLTE

SEQ ID NO: 140 - protein, 9 aa, Artificial Sequence
  goldfish TRP-1 dileucine-based sorting signal
EGRQPLLGD

SEQ ID NO: 141 - protein, 9 aa, Homo sapiens
EANQPLLTD

SEQ ID NO: 142 - protein, 9 aa, Artificial Sequence
  chicken TRP-1 dileucine-based sorting signal
ELHQPLLTD

SEQ ID NO: 143 - protein, 9 aa, Artificial Sequence
  zebrafish TRP-2 dileucine-based sorting signal
REFEPLLNA

SEQ ID NO: 144 - protein, 9 aa, Homo sapiens
EEKMAILMD

SEQ ID NO: 145 - protein, 9 aa, Homo sapiens
EEKLAILSQ

SEQ ID NO: 146 - protein, 9 aa, Mus musculus
SERDVLLDE

SEQ ID NO: 147 - protein, 9 aa, Homo sapiens
SERRNLLED

SEQ ID NO: 148 - protein, 9 aa, Rattus norvegicus
DDSGDLLPG

SEQ ID NO: 149 - protein, 9 aa, Homo sapiens
SQIKRLLSE

SEQ ID NO: 150 - protein, 9 aa, Felis catus
SHIKRLLSE

SEQ ID NO: 151 - protein, 9 aa, Mus musculus
RRTPSLLEQ

SEQ ID NO: 152 - protein, 9 aa, Homo sapiens
HRTPSLLEQ

SEQ ID NO: 153 - protein, 10 aa, Rattus norvegicus
EPRGSRLLVR

SEQ ID NO: 154 - protein, 19 aa, Homo sapiens
MDDQRDLISNNEQLPMLGR

SEQ ID NO: 155 - protein, 19 aa, Mus musculus
MDDQRDLISNHEQLPILGN

SEQ ID NO: 156 - protein, 20 aa, Artificial Sequence
  chicken Ii dileucine-based sorting signal
MAEEQRDLISSDGSSGVLPI

SEQ ID NO: 157 - protein, 23 aa, Artificial Sequence
  zebrafish Ii dileucine-based sorting signal
MEPDHQNESLIQRVPSAETILGR

SEQ ID NO: 158 - protein, 23 aa, Artificial Sequence
  zebrafish Ii dileucine-based sorting signal
MSSEGNETPLISDQSSVNMGPQP

SEQ ID NO: 159 - protein, 24 aa, Artificial Sequence
  Trypanosome Lamp dileucine-based sorting signal
RPRRRTEEDELLPEEAEGLIDPQN

SEQ ID NO: 160 - protein, 19 aa, Homo sapiens
PDKHSLLVGDFREDDDTAL

SEQ ID NO: 161 - protein, 9 aa, Homo sapiens
TERERLLNF

SEQ ID NO: 162 - protein, 8 aa, Homo sapiens
VETDDLIL

SEQ ID NO: 163 - protein, 7 aa, Caenorhabditis elegans
FENDSLL

SEQ ID NO: 164 - protein, 9 aa, Saccharomyces cerevisiae
NEQSPLLHN

SEQ ID NO: 165 - protein, 8 aa, Saccharomyces cerevisiae
SEQTRLVP

SEQ ID NO: 166 - protein, 8 aa, Saccharomyces cerevisiae
EVDLDLLK

SEQ ID NO: 167 - protein, 5 aa, Artificial Sequence
  dileucine-based sorting signal
  misc_feature (2)..(3): Xaa can be any naturally occurring amino acid
DXXLL

SEQ ID NO: 168 - protein, 13 aa, Homo sapiens
SFHDDSDEDLLHI

SEQ ID NO: 169 - protein, 13 aa, Bos taurus
TFHDDSDEDLLHV

SEQ ID NO: 170 - protein, 13 aa, Artificial Sequence
  rabbit CI-MPR dileucine-based sorting signal
SFHDDSDEDLLNI

SEQ ID NO: 171 - protein, 13 aa, Artificial Sequence
  chicken CI-MPR dileucine-based sorting signal
SFHDDSDEDLLNV

SEQ ID NO: 172 - protein, 13 aa, Homo sapiens
EESEERDDHLLPM

SEQ ID NO: 173 - protein, 13 aa, Artificial Sequence
  chicken CD-MPR dileucine-based sorting signal
DESEERDDHLLPM

SEQ ID NO: 174 - protein, 12 aa, Homo sapiens
GYHDDSDEDLLE

SEQ ID NO: 175 - protein, 13 aa, Homo sapiens
ITGFSDDVPMVIA

SEQ ID NO: 176 - protein, 13 aa, Artificial Sequence
  Hydra Head-activator BP dileucine-based sorting signal
INRFSDDEPLVVA

SEQ ID NO: 177 - protein, 13 aa, Homo sapiens
MLEASDDEALLVC

SEQ ID NO: 178 - protein, 13 aa, Homo sapiens
KNETSDDEALLLC

SEQ ID NO: 179 - protein, 12 aa, Mus musculus
WVVEAEDEPLLA

SEQ ID NO: 180 - protein, 12 aa, Homo sapiens
WVAEAEDEPLLT

SEQ ID NO: 181 - protein, 12 aa, Homo sapiens
HDDFADDISLLK

SEQ ID NO: 182 - protein, 13 aa, Mus musculus
GRDSPEDHSLLVN

SEQ ID NO: 183 - protein, 12 aa, Peromyscus maniculatus
VRCHPEDDRLLG

SEQ ID NO: 184 - protein, 13 aa, Homo sapiens
HRVSQDDLDLLTS

SEQ ID NO: 185 - protein, 13 aa, Homo sapiens
ASVSLLDDELMSL

SEQ ID NO: 186 - protein, 13 aa, Homo sapiens
ASSGLDDLDLLGK

SEQ ID NO: 187 - protein, 13 aa, Homo sapiens
VQNPSADRNLLDL

SEQ ID NO: 188 - protein, 13 aa, Homo sapiens
NALSWLDEELLCL

SEQ ID NO: 189 - protein, 13 aa, Drosophila sp.
TVDSIDDVPLLSD

SEQ ID NO: 190 - protein, 13 aa, Mus musculus
QEECPSDSEEDEG

SEQ ID NO: 191 - protein, 13 aa, Mus musculus
RDRDYDEDDEDDI

SEQ ID NO: 192 - protein, 15 aa, Mus musculus
LDETEDDELEYDDES

SEQ ID NO: 193 - protein, 10 aa, Homo sapiens
KDPDEVETES

SEQ ID NO: 194 - protein, 13 aa, Homo sapiens
HEFQDETDTEEET

SEQ ID NO: 195 - protein, 14 aa, Homo sapiens
QEKEDDGSESEEEY

SEQ ID NO: 196 - protein, 9 aa, Artificial Sequence
  VMAT1 acidic cluster sorting signal
GEDEESESD

SEQ ID NO: 197 - protein, 12 aa, Artificial Sequence
  VAMP4 acidic cluster sorting signal
GEDSDEEPDHEE

SEQ ID NO: 198 - protein, 13 aa, Herpes virus 3
FEDSESTDTEEEF

SEQ ID NO: 199 - protein, 9 aa, Human immunodeficiency virus type 1
LEAQEEEEV

SEQ ID NO: 200 - protein, 22 aa, Saccharomyces cerevisiae
ADDLESGLGAEDDLEQDEQLEG

SEQ ID NO: 201 - protein, 12 aa, Saccharomyces cerevisiae
TEIDESFEMTDF

SEQ ID NO: 202 - protein, 18 aa, Saccharomyces cerevisiae
TEPEEVEDFDFDLSDEDH

SEQ ID NO: 203 - protein, 15 aa, Saccharomyces cerevisiae
FEIEEDDVPTLEEEH

SEQ ID NO: 204 - protein, 6 aa, Artificial Sequence
  intracellular retention signal
  MISC_FEATURE (1)..(1): Xaa may be Asp or Glu
  misc_feature (2)..(4): Xaa can be any naturally occurring amino acid
  MISC_FEATURE (6)..(6): Xaa may be Leu or Ile
XXXXLX

SEQ ID NO: 205 - protein, 3 aa, Artificial Sequence
  intracellular retention signal
  MISC_FEATURE (3)..(3): Xaa may be Phe or Trp
DPX

SEQ ID NO: 206 - protein, 5 aa, Artificial Sequence
  intracellular retention signal
  misc_feature (2)..(2): Xaa can be any naturally occurring amino acid
  misc_feature (4)..(4): Xaa can be any naturally occurring amino acid
FXDXF

SEQ ID NO: 207 - protein, 5 aa, Artificial Sequence
  intracellular retention signal
  MISC_FEATURE (2)..(2): Xaa is an amino acid with a bulky hydrophobic side chain
  misc_feature (3)..(3): Xaa can be any naturally occurring amino acid
  MISC_FEATURE (4)..(4): Xaa is an amino acid with a bulky hydrophobic side chain
  MISC_FEATURE (5)..(5): Xaa may be Asp or Glu
LXXXX

SEQ ID NO: 208 - protein, 5 aa, Artificial Sequence
  intracellular retention signal
LLDLL

SEQ ID NO: 209 - protein, 5 aa, Artificial Sequence
  intracellular retention signal
PWDLW

SEQ ID NO: 210 - protein, 4 aa, Artificial Sequence
  intracellular retention signal
HDEL

SEQ ID NO: 211 - protein, 4 aa, Artificial Sequence
  membrane proximal buffer sequence
KPDK

SEQ ID NO: 212 - protein, 394 aa, Artificial Sequence
  membrane-bound DcR3 sequencehuman DcR3-CD8 stalk/TM/rigid linker
METDTLILWVLLLLVPGSTGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVHSDPTTTPAPRPPTPAPTIASQPLSLRPEACRPAAGGAVHTRGLDFACDIYIWAPLAGTCGVLLLSLVITLYCRKKRSLEAEAAAKEAAAKEAAAKEAAAKALE

SEQ ID NO: 213 - protein, 575 aa, Artificial Sequence
  bicistronic construct co-expressing an anti-Fas KDEL with FasL Anti-Fas-KDEL-2A-FasL
MGWSCIILFLVATATGVHSDIQMTQSPSSLSASVGDRVTITCRASQGIRRWLAWYQQKPEKAPKSLIYAASSLQSGVPSRFSGSGSGTDFTLTISSLQPEDFATYYCQQYNSYPYTFGQGTKLEIKRSGGGGSGGGGSGGGGSQVQLQQWGAGLLKPSETLSLICAVYGGSFSTYYWTWIRQPPGKGLEWIGEINHRGTTNYSPSLKSRVTISVDTSKNHISLNLTSVTAADTALYYCARGLLWIGEGDYGLDVWGQGTTVTVSSDPAEPSEKDELEGRGSLLTCGDVEENPGPMQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

(19) United States
(12) Patent Application Publication
(10) Pub. No.: US 2023/0133554 A1
(43) Pub. Date: May 4, 2023

(54) MOLECULE

(71) Applicant: AUTOLUS LIMITED, London (GB)

(72) Inventors: Callum McKenzie, London (GB); Shaun Cordoba, London (GB); Martin Pulé, London (GB)

(21) Appl. No.: 17/915,689

(22) PCT Filed: Apr. 8, 2021
(86) PCT No.: PCT/GB2021/050865

§ 371 Date: Sep. 29, 2022

(30) Foreign Application Priority Data
Apr. 9, 2020 (GB) 2005331.0
Nov. 2, 2020 (GB) 2017358.9
Feb. 3, 2021 (GB) 2101491.5

(51) Int. Cl. C07K 14/725; C07K 14/705; C12N 5/00

(52) U.S. Cl. CPC C07K 14/7051; C07K 14/70578; C12N 5/0081

(57) ABSTRACT

The present invention provides a method for selecting for cells transduced to express a nucleic acid sequence of interest (NOI), which comprises the following steps: (a) transducing a population of cells with a vector co-expressing the NOI and a nucleic acid sequence which inhibits Fas expression or activity in the cell; (b) exposing the cells from (a) to FasL such that untransduced cells are eliminated by apoptosis. The present invention also provides a molecule which comprises a Fas-binding domain linked to an intracellular retention signal which may be used in such a method to inhibit Fas expression.

### FIELD OF THE INVENTION

The present invention relates a method for selecting cells based on their resistance to elimination by apoptosis when exposed to Fas ligand (FasL). The present invention also relates FasL expressing cells and their use to eliminate alloreactive T-cells and other Fas-expressing cells such as cancer cells.

### BACKGROUND TO THE INVENTION

Adoptive immunotherapy of cancer involves the ex vivo generation of cancer-antigen specific cells and their administration. Adoptively transferred immune effector cells also activate existing adaptive and innate immune cells within the tumour once they activate and start causing inflammation.

The native specificity of immune effector cells can be exploited in adoptive immunotherapy—for example during the generation of melanoma specific T-cells from expansion of tumour infiltrating lymphocytes in tumour resections. Otherwise a specificity can be grafted onto a T-cell using genetic engineering. Two common methods for achieving this are using chimeric antigen receptors (CARs) or transgenic T-cell receptors (TCRs).

Adoptive immunotherapy has been successful in treating a number of lymphoid malignancies, such as B-cell Acute Lymphoblastic Leukaemia (B-ALL), Diffuse Large B-cell Lymphoma (DLBCL) and Multiple Myeloma (MM).

Purification of Transduced Cells

Engineered immunotherapy products, such as CAR- or engineered TCR-expressing cells are typically made by transduction with an integrating vector, such as a lentiviral vector or a retroviral vector.

Transduction is never one hundred percent efficient, meaning that the patient receives a mixture of transduced and untransduced cells.

It is preferable to administer patients with cell therapy products that are composed predominately of transduced cells as it makes the product more effective and efficient, and in some cases, safer.

One option to increase the proportion of transduced cells in the composition is to purify the cell composition post-transduction, for example by flow cytometry or using a magnetic bead column. However, this increases manufacturing costs and complexity, and can be difficult to scale up to a commercial scale manufacturing process. These procedures also place the product under stress which may affect, for example, the exhaustion and/or differentiation state of the cells in the composition.

These problems are compounded in situations where cells are transduced with more than one vector. Cell therapy products often require transductions with two or more separate vectors. For example, this can be required in situations where a CAR is expressed in a cell with one or more modules (e.g. module a′, b′, c′, d′) for example, a suicide gene, marker protein, cytokine, secreted antibody, or module conferring resistance to checkpoint inhibition or TGFβ inhibition. Where multiple or genetically “large” modules are required, it is likely to be impossible to encode everything on a single vector due to physical limits in DNA size.

In this situation it is important that the all of the transduced cells administered to the patients express the CAR polypeptide. Administration of transduced cells which no not express the CAR (i.e. cells which just express one or more modules) would compromise the efficacy of the overall product and may be unsafe.

There are broadly two approaches to ensure all transduced cells in a double, triple or multi-vector transduction have the expression of the CAR. The first approach is to encode for the CAR polypeptide on every vector used in the multiple vector transduction. This requires a duplication of the CAR sequence on every vector used in the multiple transduction which limits space on each vector.

The second approach is to express the CAR and a protein marker from the first vector (vector A) then have the other vector(s) absent of the CAR and marker. After transduction with both or multiple vectors, the product will need to be purified by, for example, flow cytometry sorting or using magnetic bead column, which suffers from the disadvantages mentioned above.

There is therefore a need for alternative method for purifying transduced cells prior to adoptive immunotherapy.

Heterogeneity of Antigen Expression

Tumour heterogeneity describes the observation that different tumour cells can show distinct morphological and phenotypic profiles, including cellular morphology, gene expression, metabolism, motility, proliferation, and metastatic potential.

Heterogeneity occurs between patients, between tumours (inter-tumour heterogeneity) and within tumours (intra-tumour heterogeneity). Multiple types of heterogeneity have been observed between tumour cells, stemming from both genetic and non-genetic variability.

Heterogeneity in target antigen in and between tumours poses a particular problem for cellular immunotherapy approaches such as those using CAR- or engineered TCR-expressing T cells.

By their very nature, such therapies are designed to be specific to a particular antigen and, where a cancer cell does not express that antigen, it will escape undetected by the CAR or TCR.

A related problem is provided by the Goldie-Coldman hypothesis: which describes that the sole targeting of a single antigen may result in tumour escape by modulation of said antigen due to the high mutation rate inherent in most cancers. For example, a problem with immunotherapeutics targeted against CD19 is that a B-cell malignancy may mutate and become CD19-negative. This may result in relapse with CD19-negative cancers which are not responsive to CD19 targeted therapeutics. For example, in one paediatric study, Grupp et al. reported that half of all relapses following CD19-targeted chimeric antigen receptor therapy for B-acute Lymphoblastic leukaemia (B-ALL) were due to CD19-negative disease (56th American Society of Hematology Annual Meeting and Exposition).

It is possible to mitigate the risks of tumour heterogeneity and target-antigen negative escape by targeting more than one antigen, for example, by using a Tandem CAR (or “TanCAR”) or a cell which expresses two or more separate CARs, each targeting a different antigen.

However, both of these approaches increase the complexity and size of the CAR-encoding cassette. They also rely on the existence of a suitable “back-up” antigen which will be expressed by effectively all cancer cells even if they are or become negative for the first antigen. Such an antigen may be difficult to find for solid cancers and even some liquid cancers, such as AML.

There is therefore a need for alternative approaches to address the issues of antigen heterogeneity and antigen-negative escape with CAR- and engineered T-cell approaches.

Allogeneic Approach

In hematopoietic stem cell transplants (HSCT), mismatches in HLA between recipient and donor can lead to rejection of the graft by host immune cells (HvG) or graft-vs-host disease (GVHD). Alloreactive T-cells that recognise mismatches HLA via their T-cell receptor (TCR) are major mediators of rejection and GVHD.

When CAR T-cells or engineered TCR-expressing T cells are generated from autologous cells, allo-responses do not occur. However, in some circumstances, it is necessary or desirable to use T-cells from an allogeneic donor, for example where the patient has had an allogeneic haematopoietic stem cell transplant, or has insufficient T-cells to generate a CAR T-cell product due to chemotherapy induced lymphopenia.

Various strategies have been described to reduce or avoid HvG and GVH diseases by tampering with the TCR:HLA interaction. These include strategies to block expression of the TCR and/or HLA at the genetic level or at the cell surface. Whichever method is chosen, the cells need to be highly purified so that only transduced cells with the necessary modifications are administered to the patient, otherwise non-transduced cells that have not been modified will cause GvHD.

Host vs graft disease is a particular problem for CAR-mediated approaches to treat T-cell malignancies. WO2015/132598 describes a method whereby it is possible to deplete malignant T-cells in a subject using CAR-T cells which specifically bind TCR beta constant region 1 (TRBC1) or TRBC2.

As this approach involves the specific binding of a T-cell receptor on a target T-cell, the targeted T-cell can “fight back” due to ligation of its TCR, resulting in depletion of the grafted/desirable T-cells.

There is therefore a need for alternative approached to prevent graft rejection, GVHD and target “fight-back” of malignant T cells.

Avoiding Fas/Fas-L Mediated Killing of Immune Effector Cells

The tumour microenvironment (TME) provides tumour cells with essential signals for survival, growth and immune resistance. The TME is therefore immunosuppressive and can inhibit the persistence and survival of immune cells in cancer immunotherapies, such as CAR T cell therapy. Immunosuppressive mechanisms employed by the TME include e.g., upregulating immune checkpoint signals such as PDL-1 and/or CTLA-4 and secretion of cytokines such as IL-6 and/or TGF-beta.

Recent evidence suggests that immunosuppressive TME also upregulates death ligands such as Fas ligand (FasL). FasL induces apoptosis of immune cells that express death receptors for Fas, such as tumour-infiltrating lymphocytes (TILs).

In addition to the TME upregulating death ligands, it has also been shown that CAR-T cells themselves upregulate death receptors and their ligands upon activation and transduction of the CAR construct, triggering activation-induced death (AICD) and further exacerbating the problem of CAR-T cell persistence in the TME. Moreover, FasL is not only expressed by activated T cells bit is also upregulated by exposure to IFNγ that is produced by activated T cells.

Notably, third generation CARs, which have two co-stimulatory endodomains, seem to be particularly susceptible to AICD as a result of increased FasL expression (Xu et al., 2017, Hum Vaccin Immunother. 13(7):1548-1555, Benmebarek et al., 2019, In J Mol Sci. 20(6): 1283).

In addition to inducing apoptosis, Fas signals via non-apoptotic cascades and it has been shown that when naive and memory T cells are mixed prior to adoptive transfer, naïve T cells undergo precocious differentiation which limits their anti-tumour efficacy. This effect is mediated by non-apoptotic, AKT-driven Fas signalling on memory CD8 T cells.

There is thus a need for mechanisms to prevent FasL-induced death and improve the effectiveness of engineered immune effector cells to persist and survive in the TME.

### DESCRIPTION OF THE FIGURES

FIG. 1—(A) Peripheral blood mononuclear cells (PBMCs) were either non-transduced (NT) or transduced to express either the anti-CD19 CAR (Fmc63-41BBz) or co-express, via a 2A self-cleaving peptide, FasL, truncated Fas (FasΔDD), or FasΔDD and FasL. The percentage of cells expressing the constructs were analysed by flow cytometry, one and four days after transduction, by staining for an independent marker, RQR8. The flow plots show gating for RQR8 positive PBMCs, with RQR8 on the x-axis and forward scatter on the y-axis. Flow plots are representative of one PBMC donor. (B) PBMCs were treated as described in (A) and the percentage difference of RQR8-expressing PBMCs four days after transduction versus one day after transduction was calculated. Lines represent the mean value of four independent PBMC donors, with the error bars representing standard deviation.

FIG. 2—PBMCs were transduced on day 0 to express either the anti-CD19 (Fmc63) CAR (circles) or co-express, via a 2A self-cleaving peptide, FasL (squares), truncated Fas (FasΔDD, upward triangles) or FasΔDD and FasL (downward triangles). The day after transduction (day 1) equal numbers of cells were seeded into wells of a 96-well plate and cultured in the presence of IL-2. Absolute cell counts of transduced cells were calculated at day 1 and day 4 by flow cytometry analysis using counting beads. Transduced cells were detected through expression of an independent marker, RQR8. Lines represent the mean value of eight independent PBMC donors, with the error bars representing standard deviation.

FIG. 3—Recombinant FasL was immobilised into wells of a 96-well cell culture plate overnight at 4° C. (0, 0.1 or 1 μg per well). The following day, the FasL-immobilised cell culture plate was washed four times with PBS and PBMCs expressing either the anti-CD19 (fmc63) CAR (circles) or co-expressing truncated Fas (FasΔDD, squares) were seeded into wells and cultured for five days. Absolute cell counts of transduced cells were analysed by flow cytometry using counting beads, with transduced cells detected through expression of an independent marker, RQR8. Cell counts were made relative to PBMCs seeded in the absence of recombinant FasL. Lines represent the mean value of three independent PBMC donors, with the error bars representing standard deviation.

FIG. 4—PBMCs were transduced to co-express via a 2A self-cleaving peptide an anti-CD19 CAR and FasL. At the point of transduction, cells were either treated with an isotype control (squares) or an anti-Fas blocking antibody (circles) at the stated concentrations. Absolute viable cell counts of PBMCs were calculated three days after transduction. Two independent donors were analysed.

FIG. 5—PBMCs were either non-transduced (NT) or transduced to co-express via a 2A self-cleaving peptide a marker gene, RQR8, and an anti-CD19 CAR (Fmc63-CD3z), or additionally express chimeric Fas receptors, Fas-CD40 (Fas ectodomain, Fas transmembrane domain, CD40 endodomain) or Fas-XEDAR (Fas ectodomain, Fas transmembrane domain, XEDAR endodomain), or truncated Fas (FasΔDD). Equal numbers of PBMCs were seeded into wells of a 96-well plate in the presence or absence of multimeric FasL (MEGA FasL, 100 ng/ml) and then incubated for three days, at which point cells were analysed by flow cytometry, staining for the Fmc63-CD3z CAR (A) and Fas (B).

FIG. 6—PBMCs either non-transduced (NT) or transduced to express either the anti-CD19 CAR (aCD19 Fmc63, squares) or co-express via a 2A self-cleaving peptide, truncated Fas (FasΔDD, upward triangles), or FasΔDD and FasL (downward triangles), were co-cultured with Nalm-6, Raji, and high and low density CD19-expressing SupT1 cell lines at a 1:8 effector to target ratio for 72 hours. Target cell killing was quantified by flow cytometry and normalised to NT PBMCs. Lines represent the mean value of three independent PBMC donors, with the error bars representing standard deviation.

FIG. 7—Annotated sequence for an example of a membrane-bound DcR3 sequence: Human DcR3-CD8 stalk/TM/rigid linker

FIG. 8—Annotated sequence for an example of a bicistronic construct co-expressing an anti-Fas KDEL with FasL: Anti-Fas-KDEL-2A-FasL

FIG. 9—Schematic diagram showing Fas/FasL-mediated induction of apoptosis

FIG. 10—Schematic diagrams illustrating (A) T-cell co-culture assay (co-expression of dFas and FasL) and (B) Immobilised FasL assay

FIG. 11—Schematic diagrams illustrating (A) Host v Graft (i.e. host T cell attacking CAR-T cell); (B) Preventing Host v Graft by the inhibition of Fas expression (dFas*) and the expression of FasL by the CAR-T cell; and (C) CAR-T mediated killing of antigen-positive cancer cells, via the CAR; and antigen negative cancer cells, via FasL. *Inhibition of Fas is shown as truncated Fas lacking the Death domain, but it could equally be due to an alternative dominant negative Fas receptor, Fas knockout, knockdown, or intracellular retention

FIG. 12—Schematic diagram illustrating the intracellular retention of Fas using an anti-Fas-KDEL molecule. (A) The Fas receptor is synthesised at the rough endoplasmic reticulum (ER) and transported to the plasma membrane via the Golgi apparatus. (B) The anti-Fas-KDEL polypeptide binds to the extracellular domain on Fas and consequently Fas is retained at the ER via the KDEL receptor recognising the KDEL sequence. Fas molecules that are trafficked to the Golgi apparatus (to eventually be transported to the plasma membrane) are trafficked back to the ER by retrograde transport via the KDEL receptor recognising the KDEL sequence.

FIG. 13—Fas staining histograms of PBMCs and SupT1 cells transduced with the Fas binder-KDEL polypeptide demonstrating decreased staining of Fas when transduced with the Fas binder-KDEL polypeptide.

FIG. 14—Two independent PBMC donors and SupT1 cells that were either non-transduced (NT) or transduced to express a Fas binder-KDEL polypeptide, were either untreated or treated with MEGA FasL (100 ng/mL) for 48 hours, at which point cells were analysed by flow cytometry. The raw flow cytometry plots are shown in (A), with the percentage of surviving BFP positive PBMCs and SupT1s made relative to untreated conditions shown in (B).

FIG. 15—Two independent PBMC donors that were either non-transduced (NT) or transduced to express a Fas binder-KDEL polypeptide, were co-cultured with NT SupT1 cells or FasL-expressing SupT1 cells at a 1:2 PBMC:SupT1 ratio for 48 hours, at which point cells were analysed by flow cytometry. Raw flow cytometry plots are shown in (A) with PBMCs identified from SupT1 cells through dual expression of CD2 and CD3. The percentage of surviving BFP positive PBMCs made relative to NT SupT1 cells are shown in (B).

### SUMMARY OF ASPECTS OF THE INVENTION

The present inventors have developed a method for screening for transduced cells depending on the ability of such cell to resist Fas-mediated apoptosis.

The inventors have also developed FasL expressing cells capable of “self-selecting” for transduced cells and capable of eliminating alloreactive T-cells and other Fas-expressing cells such as cancer cells.

Thus, in a first aspect, the present invention provides a method for selecting for cells transduced to express a nucleic acid sequence of interest (NOI), which comprises the following steps:
(a) transducing a population of cells with a vector co-expressing (i) the NOI and (ii) a nucleic acid sequence which inhibits Fas expression or activity in the cell;
(b) exposing the cells from (a) to FasL.
   Untransduced cells are thereby eliminated by apoptosis.

The nucleic acid sequence which protects the cell from FasL-induced apoptosis may inhibit Fas expression in the cell. For example, the nucleic acid sequence may be selected from the following group:
(i) dominant negative Fas;
(ii) molecule which comprises a Fas-binding domain linked to an intracellular retention signal;
(iii) a gRNA molecule comprising a targeting domain that is complementary with a target domain in the Fas gene; or
(iv) an siRNA complementary with a target domain in the Fas gene.

The vector may comprise a nucleic acid sequence encoding dominant negative Fas (dnFas), which dnFas comprises the Fas extracellular domain but has a truncated or mutated death domain so that it does not bind FADD.

Dominant negative Fas may comprise the Fas extracellular domain and an endodomain from a TNF receptor, for example an endodomain from decoy receptor 2 (DcR2), GITR, CD30, XEDAR, CD40, CD27, HVEM, BCMA, 4-1BB or Fn14.

Alternatively, the nucleic acid sequence which protects the cell from FasL-induced apoptosis may encode a molecule which competitively inhibits Fas binding to FasL. For example, the nucleic acid sequence may encode membrane-bound DcR3.

Membrane-bound DcR3 may be a transmembrane protein comprising a DcR3 extracellular domain and a stimulatory endodomain. The stimulatory endodomain may be a co-stimulatory domain such as 41BB or CD28 endodomain.

In step (b) of the method of the first aspect of the invention, FasL may be soluble FasL, FasL bound to a solid substrate, or FasL expressed on the surface of a cell.

Hexameric FasL may be used in step (b).

The method of the invention may comprise the following steps:
(a) transducing a population of cells with a plurality of vectors, one of which co-expresses the NOI and the nucleic acid sequence which inhibits Fas expression or activity in the cell;
(b) exposing the cells from (a) to FasL.
Cells transduced with the vector expressing the NOI or cells transduced with a combination of vectors including the vector expressing the NOI are thereby selected as they are protected from apoptosis.

The method of the invention may comprise the following steps:
(a) transducing a population of cells with a vector co-expressing (i) the NOI, (ii) nucleic acid sequence which inhibits Fas expression or activity in the cell and (iii) a nucleic acid sequence encoding FasL;
(b) culturing the cells.

Cells transduced with the vector are thereby “self-selected” as they are protected from apoptosis.

The method of the invention may comprise the following steps:
(a) co-transducing a population of cells with:
   a first vector co-expressing (i) a CAR or TCR; and (ii) the nucleic acid sequence which inhibits Fas expression or activity in the cell; and
   a second vector expressing FasL
(b) culturing the cells.

Cells transduced with the first vector, together with cells co-transduced with the first and second vector are thereby selected as they are protected from apoptosis.

The second vector may also comprise a nucleic acid sequence encoding a protein of interest, such as a cytokine. The cytokine may, for example, be IL-12.

The cell may express mutant FasL with reduced susceptibility to cleavage. For example, mutant FasL may have reduced susceptibility to metalloprotease cleavage. In this respect, mutant FasL may comprise a deletion of amino acid residues 126-135 with reference to the sequence shown as SEQ ID No. 7.

The cell may express FasL which comprises a signalling domain and/or a costimulatory domain. For example, the cell may express one of the following: FasL-CD3z; FasL-CD28; FasL-41BB, FasL-CD28z; FasL-41BBz.

The NOI expressed by the vector in step (a) of the method of the invention may encode any protein of interest. It may, for example, encode a chimeric antigen receptor (CAR) or a transgenic T cell receptor (TCR).

Alternatively, the NOI expressed by the vector in step (a) of the method of the invention may inhibit expression of endogenous T-cell receptor (TCR). In this respect, the NOI may encode:
(i) a TCR or CD3-binding domain linked to an intracellular retention signal;
(ii) a gRNA molecule comprising a targeting domain that is complementary with a target domain in a TCR gene; or
(iii) an siRNA complementary with a target domain in a TCR gene.

The second aspect of the invention relates to a cell having reduced expression or activity of Fas.

The second aspect of the invention provides a cell engineered to express (a) a nucleic acid sequence which inhibits Fas expression or activity in the cell; and (b) FasL

The nucleic acid sequence which inhibits Fas expression or activity in the cell may, for example:
(i) encode dominant negative Fas;
(ii) encode a molecule which comprises a Fas-binding domain linked to an intracellular retention signal;
(iii) be a gRNA molecule comprising a targeting domain that is complementary with a target domain in the Fas gene;
(iv) be an siRNA complementary with a target domain in the Fas gene; or
(v) encode membrane-bound DcR3.

The cell may also express
(c) a chimeric antigen receptor (CAR) or a transgenic T cell receptor (TCR) and/or a nucleic acid sequence of interest (NOI) which inhibits expression of endogenous T-cell receptor (TCR).

Where the cell expresses a chimeric antigen receptor (CAR) or a transgenic T cell receptor (TCR), the CAR or TCR-expressing nucleic acid may be introduced into the Fas gene, knocking-out Fas gene expression.

FasL expressed by the cell may be mutant FasL, having reduced susceptibility to cleavage, or FasL which comprises a signalling domain and/or a costimulatory domain as described above in connection with the first aspect of the invention.

The cell may co-express β2-microglibulin (β2m) linked to a signalling domain and/or a costimulatory domain. For example, the cell may express one of the following: β2m-CD3z; β2m-CD28; 132m-41BB.

The cell may be engineered to be resistant to one or more calcineurin inhibitors. In this respect the cell may express:
calcineurin A comprising mutations T351E and L354A with reference to the shown as SEQ ID No. 71;
calcineurin A comprising mutations V314R and Y341F and with reference to shown as SEQ ID No. 71; or
calcineurin B comprising mutation L124T and K-125-LA-Ins with reference to shown as SEQ ID No. 72.

The third aspect of the invention provides a nucleic acid construct for expression in a cell which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a nucleic acid sequence which inhibits Fas expression or activity in the cell; and optionally
a nucleic acid sequence encoding FasL

In a fourth aspect, the invention provides a vector which comprises a nucleic acid construct according to the third aspect of the invention.

In a fifth aspect, the invention provides a kit of nucleic acid sequences which comprises:
a first nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a second nucleic acid sequence which inhibits Fas expression or activity when expressed in a cell; and optionally
a third nucleic acid sequence encoding FasL

In a sixth aspect, the invention provides a kit of vectors which comprises:
a first vector comprising a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a second vector comprising a nucleic acid sequence which inhibits Fas expression or activity when expressed in a cell; and optionally
a third vector comprising a nucleic acid sequence encoding FasL

In an seventh aspect, the invention provides a method for making a cell according to the second aspect of the invention which comprises the step of introducing into a cell ex vivo: a nucleic acid construct according to the third aspect of the invention, a vector according to fourth aspect of the invention, a kit of nucleic acid sequences according to the fifth aspect of the invention or a kit of vectors according to the sixth aspect of the invention.

In an eighth aspect, there is provided a pharmaceutical composition which comprises plurality of cells selected by a method according to the first aspect of the invention or a plurality of cells according to the second aspect of the invention.

In a ninth aspect, there is provided a method for treating cancer which comprises the step of administering a pharmaceutical composition according to the eighth aspect of the invention to a subject.

In a tenth aspect, there is provided the use of a plurality of cells selected by a method according to the first aspect of the invention or a plurality of cells according to the second aspect of the invention in the manufacture of a medicament for the treatment of cancer.

In an eleventh aspect, there is provided a pharmaceutical composition according to the eighth aspect of the invention for use in a method for treating cancer.

In a twelfth aspect, the invention provides a method for treating cancer in a subject which comprises cancer cells negative for CAR or TCR target antigen, which comprises the step of administering a plurality of cells according to the second aspect of the invention to the subject. Target-antigen negative cancer cells in the subject are thereby killed by FasL binding to Fas on the cancer cells.

The invention also provides the use of a plurality of cells according to the second aspect of the invention to kill target-antigen negative cancer cells in a subject.

In a thirteenth aspect the invention provides a method for depleting alloreactive immune cells from a population of immune cells, which comprises the step of contacting the population of immune cells with a plurality of cells according to the second aspect of the invention in vitro.

In a fourteenth aspect, the invention provides a method for preventing graft versus host disease in a subject, which comprises the step of administering a plurality of cells selected by a method according to the first aspect of the invention, to the subject.

The invention also provides the use of a plurality of cells selected by a method according to the first aspect of the invention for use in preventing graft versus host disease in a subject.

In a fifteenth aspect, the invention provides an allogeneic or autologous transplant which has been depleted of untransduced cells by a method according to the first aspect of the invention.

In a sixteenth aspect, there is provided a method for preventing graft rejection in a subject, which comprises the step of administering a plurality of cells according to the second aspect of the invention, to the subject.

The invention also provides the use of a plurality of cells according to the second aspect of the invention for use in preventing graft rejection in a subject.

In the method of the invention FasL is used to select for transduced cells. By linking insensitivity to Fas to transduction with a desired nucleotide of interest (NOI) is it possible to use FasL-mediated apoptosis to select for cells expressing the NOI. The presence of FasL can either be in the form of exogenous recombinant FasL or by the co-expression of FasL by the cells themselves.

The method of the invention provides a straightforward way to select or “self-select” transduced cells based on their resistance to FasL mediated apoptosis.

The method of the invention can be used to prevent graft-vs-host disease. Where the NOI inhibits expression of endogenous T-cell receptor (TCR), untransduced cells (which retain expression of the endogenous TCR) will be eliminated by apoptosis. The resulting population should be selected for cells which do not express the endogenous TCR which are safe to administer to a patient without causing GvHD.

The expression of FasL by the cell also has other advantages. For example, as many cancer cells express Fas, administration of cells co-expressing a CAR or engineered TCR and FasL provides a two-pronged method for elimination of cancer cells: target antigen-positive cancer cells may be recognised by the CAR/TCR; whereas antigen-negative cancer cells may be killed by FasL on the effector cell binding Fas on the cancer cell.

Also, in situations where the target for a CAR/TCR expressing cell is a T cell (for example in the treatment of T-cell leukemias or lymphomas) expression of FasL gives the CAR/TCR-expressing cell an advantage over the target cell, as it can kill the target call by Fas/FasL mediated apoptosis as well as CAR/TCR mediated killing.

This is also true in the prevention of graft rejection. Alloreactive T cells in a patient or cell sample can be eliminated by the cells of the invention by FasL:Fas mediated apolotosis.

### DETAILED DESCRIPTION

Fas

The present invention provides a method for selecting transduced cells based on their capacity to resist Fas-mediated apoptosis.

The present invention provides a molecule which comprises a Fas-binding domain linked to an intracellular retention signal. When this molecule is expressed in a cell, it binds newly synthesised Fas and retains it in an intracellular compartment or complex, down-regulating the cell surface expression of Fas.

The Fas receptor (also known as Fas, FasR, CD95, tumour necrosis factor receptor superfamily member 6) is a type 1 transmembrane glycoprotein receptor which is localized on the surface of various cells including lymphocytes and hepatocytes. The Fas receptor triggers a signal transduction pathway leading to apoptosis and expression of Fas can be increased by activation of lymphocytes as well as by cytokines such as IFNγ and TNF. The interaction of Fas with its ligand FasL (FasL/CD95L) regulates numerous physiological and pathological processes that are mediated through programmed cell death.

Both Fas and FasL are members of the TNF-R superfamily, all of which contain one to five extracellular cysteine rich domains (CRDs), and in their cytoplasmic tail, a death domain (DD), which are 80-100 residue long motifs.

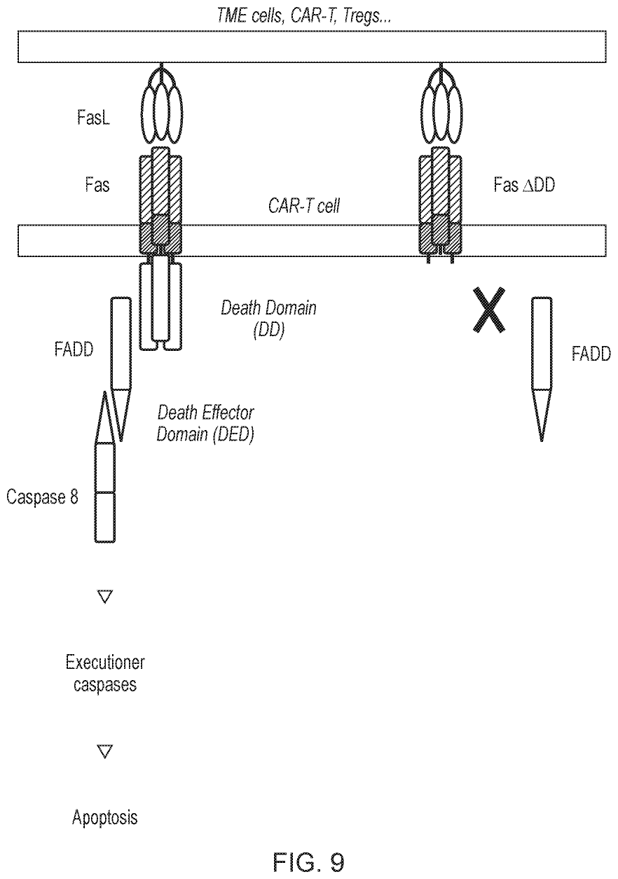

Fas binding to FasL triggers receptor trimerization and recruits a protein called Fas-associated death domain (FADD) via homotypic interactions of their death domains (DDs). In turn, FADD then recruits procaspase-8 to the activated receptor and the resulting death-inducing signalling complex (DISC) performs caspase-8 proteolytic activation, which initiates the subsequent cascade of caspases (aspartate-specific cysteine proteases) mediating apoptosis (see FIG. 9).

FasL has been reported to be expressed by many cancers themselves such as melanomas, lung carcinomas, hepatocellular carcinomas, esophageal carcinomas and colon carcinomas. Tumour endothelial cells, which line blood vessels and control blood and nutrient flow and trafficking of leukocytes, have been shown to express FasL, whereas normal vasculature do not. FasL is expressed by myeloid-derived suppressor cells (MDSC). MDSC are a heterogenous populations of cells that expand during cancer, chronic inflammation, autoimmune and infectious diseases, and dampen down the immune response thereby promoting tumour growth. FasL is also reportedly expressed by cancer-associated fibroblasts (CAFs) and CD4+CD25+ regulatory T cells.

FasL is also expressed by T cells themselves and has been shown to be further upregulated in CAR-Ts, meaning CAR T cells themselves are susceptible to fratricide. FasL is not only expressed by activated T cells but is also upregulated by exposure to IFNγ that is produced by activated T cells.

As a mechanism of T-cell homeostasis, T cells that are constantly activated die through a mechanism called activation-induced cell death (AICD), and the Fas-FasL pathway has been characterised as the cause of this. Despite improved cytolytic activity and cytokine production, third generation CAR-T cells are more susceptible to AICD as a result of increased FasL expression.

The sequence of human Fas is available from Uniprot (Accession No. P25445) and shown below as sequence ID No. 1. In this sequence, residues 26-173 form the extracellular domain (SEQ ID No. 2); residues 174-190 form the transmembrane domain (SEQ ID No. 3); and residues 191-335 form the cytoplasmic domain (SEQ ID No. 4). In SEQ ID No. 4, the portion of sequence that is deleted in the truncated Fas described in the Examples (FasΔDD) is underlined.

(human Fas)
SEQ ID No. 1
MLGIWTLLPLVLTSVARLSSKSVNAQVTDINSKGLELRKTVTTVETQ
NLEGLHHDGQFCHKPCPPGERKARDCTVNGDEPDCVPCQEGKEYTDK
AHFSSKCRRCRLCDEGHGLEVEINCTRTQNTKCRCKPNFFCNSTVCE
HCDPCTKCEHGIIKECTLTSNTKCKEEGSRSNLGWLCLLLLPIPLIV
WVKRKEVQKTCRKHRKENQGSHESPTLNPETVAINLSDVDLSKYITT
IAGVMTLSQVKGFVRKNGVNEAKIDEIKNDNVQDTAEQKVQLLRNWH
QLHGKKEAYDTLIKDLKKANLCTLAEKIQTIILKDITSDSENSNFRN
EIQSLV
(Fas extracellular domain)
SEQ ID No. 2
QVTDINSKGLELRKTVTTVETQNLEGLHHDGQFCHKPCPPGERKARD
CTVNGDEPDCVPCQEGKEYTDKAHFSSKCRRCRLCDEGHGLEVEINC
TRTQNTKCRCKPNFFCNSTVCEHCDPCTKCEHGIIKECTLTSNTKCK
EEGSRSN
(Fas TM domain)
SEQ ID No. 3
LGWLCLLLLPIPLIVWV
(Fas cytoplasmic domain)
SEQ ID No. 4
KRKEVQKTCRKHRKENQGSHESPTLNPETVAINLSDVDLSKYITTIA
GVMTLSQVKGFVRKNGVNEAKIDEIKNDNVQDTAEQKVQLLRNWHQL
HGKKEAYDTLIKDLKKANLCTLAEKIQTIILKDITDSENSNFRNEIQ
SLV

Inhibition of Fas Expression or Activity

In the cell of the present invention, the Fas expression or activity is inhibited, reduced or blocked.

The term “expression” is intended to include gene expression, protein expression and cell surface expression of Fas. Inhibition of expression includes any mechanism by which the expression of Fas at the cell surface is reduced, such as genetic editing at the genomic or RNA level, inhibition of translation or export of the Fas protein from intracellular compartment(s) to the cell surface.

The cell of the present invention comprises a nucleic acid sequence which inhibits Fas expression in the cell. The nucleic acid sequence may, for example:
(i) encode a dominant negative Fas molecule;
(ii) encode a molecule comprising Fas-binding domain linked to an intracellular retention signal;
(ii) produce a gRNA molecule that targets the Fas gene; or
(iii) produces an siRNA complementary with a target domain in the Fas gene.

Fas-KDEL

The nucleic acid sequence which inhibits Fas expression in the cell may encode a molecule comprising Fas-binding domain linked to an intracellular retention signal.

The molecule may comprise i) a binding domain which binds to a Fas extracellular domain and ii) a retention domain that intracellularly retains Fas within the endoplasmic reticulum or Golgi apparatus.

The retention domain may be C-terminal to the binding domain.

Fas Binding Domain

The Fas-binding domain may be a protein or polypeptide chain which is capable of binding to Fas.

The Fas-binding domain may comprises an antibody, an antibody fragment or antigen binding fragment, a single-chain variable fragment (scFv), a domain antibody (dAb), a Fab antigen binding domain (Fab), a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, an abdurin/nanoantibody, a centyrin, an alphabody or a nanofitin which binds to Fas.

In particular, the Fas-binding domain may be or comprise a domain antibody (dAb) or a single-chain variable fragment (scFv).

A single domain antibody may, for example, be derived from an artificial VHH fragment or a camelid antibody

The Fas-binding domain may bind the extracellular domain of Fas. The target-binding domain may bind the sequence shown as SEQ ID No. 2.

Apoptosis is induced when FasL binds to the Fas receptor. An anti-Fas retaining polypeptide which binds to the extracellular domain of Fas has the additional advantage that, in addition to downregulating the expression of Fas at the cell surface, it also directly blocks the interaction of FasL to Fas for any residual Fas expressed at the cell surface inhibiting the induction of apoptosis.

Numerous Fas-binding antibodies are known in the art. For example, WO2010/102792 describes the anti-Fas antibody F45D9. The VH and VL domains from this antibody are shown below as SEQ ID No. 5 and 6 respectively.

VH domain from anti-Fas antibody
(SEQ ID No. 5)
QVQLQQWGAGLLKPSETLSLICAVYGGSFSTYYWTWIRQPPGKGLEW
IGEINHRGTTNYSPSLKSRVTISVDTSKNHISLNLTSVTAADTALYY
CARGLLWIGEGDYGLDVWGQGTTVTVSS
VL domain from anti-Fas antibody
(SEQ ID No. 6)
DIQMTQSPSSLSASVGDRVTITCRASQGIRRWLAWYQQKPEKAPKSL
IYAASSLQSGVPSRFSGSGSGTDFTLTISSLQPEDFATYYCQQYNSY
PYTFGQGTKLEIKR

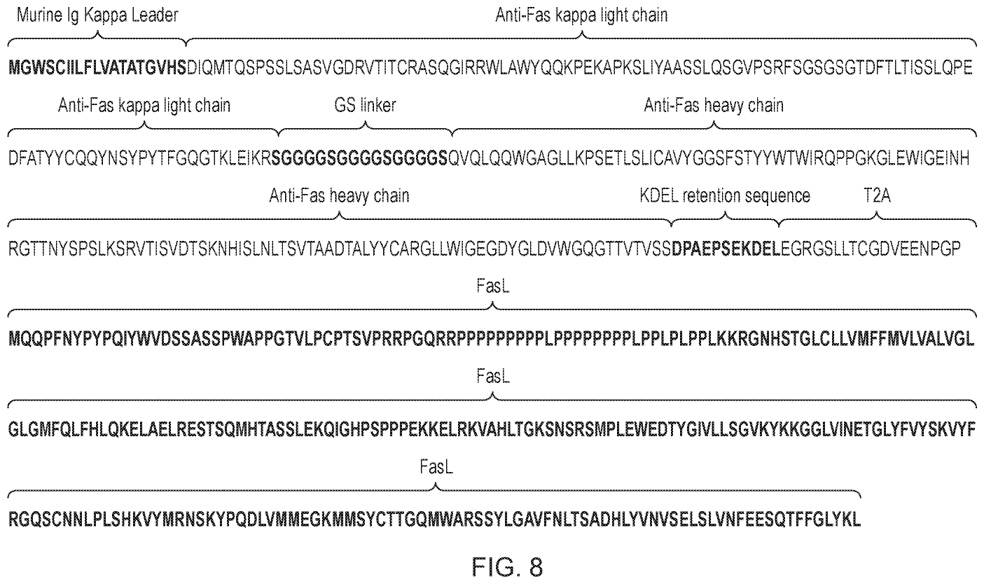

A construct sequence which comprises the sequence of an anti-Fas KDEL having an F45D9 scFv Fas-binding domain is shown in FIG. 8. In this construct, the Fas-KDEL is co-expressed with FasL.

The Fas-binding domain may comprise an antibody or fragment thereof which binds Fas, such as an scFv comprising the VH and VL sequences shown as SEQ ID No. 5 and 6.

Alternatively, the Fas-binding domain may comprise all or part of Fas ligand (FasL), the natural ligand for Fas. Human FasL has the sequence shown as SEQ ID No. 7. The Fas-binding domain may comprise all of FasL or a portion thereof. The Fas-binding domain may comprise FasL, FasL extracellular domain (shown below as SEQ ID No. 8) or a variant or truncated portion thereof which retains the capacity to bind Fas.

(human FasL)
SEQ ID No. 7
MQQPFNYPYPQIYWVDSSASSPWAPPGTVLPCPTSVPRRPGQRRPPP
PPPPPPLPPPPPPPPLPPLPLPPLKKRGNHSTGLCLLVMFFMVLVAL
VGLGLGMFQLFHLQKELAELRESTSQMHTASSLEKQIGHPSPPPEKK
ELRKVAHLTGKSNSRSMPLEWEDTYGIVLLSGVKYKKGGLVINETGL
YFVYSKVYFRGQSCNNLPLSHKVYMRNSKYPQDLVMMEGKMMSYCTT
GQMWARSSYLGAVFNLTSADHLYVNVSELSLVNFEESQTFFGLYKL
(FasL extracellular domain)
SEQ ID No. 8
ALVGLGLGMFQLFHLQKELAELRESTSQMHTASSLEKQIGHPSPPPE
KKELRKVAHLTGKSNSRSMPLEWEDTYGIVLLSGVKYKKGGLVINET
GLYFVYSKVYFRGQSCNNLPLSHKVYMRNSKYPQDLVMMEGKMMSYC
TTGQMWARSSYLGAVFNLTSADHLYVNVSELSLVNFEESQTFFGLYK
L

The molecule may comprise the Fas-binding site of the FasL extracellular domain. A variant FasL extracellular domain may have 80%, 85%, 90%, 95% or 99% identity with the sequence shown as SEQ ID No. 8, provided that the variant sequence retains the capacity to bind Fas.

FasL is a type II transmembrane protein, so the C-terminus is extracellular. The molecule may comprise a signal sequence on the N-terminus, followed by the FasL ectodomain, then an intrcellular retention sequence (such as a KDEL-containing sequence) on the C-terminus.

Intracellular Retention Signal

Protein targeting or protein sorting is the biological mechanism by which proteins are transported to the appropriate destinations in the cell or transported out of the cell. Proteins can be targeted to the inner space of an organelle, different intracellular membranes, plasma membrane, or to exterior of the cell via secretion. This delivery process is carried out based on sequence information contain in the protein itself.

Proteins synthesised in the rough endoplasmic reticulum (ER) of eukaryotic cells use the exocytic pathway for transport to their final destinations. Proteins lacking special sorting signals are vectorially transported from the ER via the Golgi and the trans-Golgi network (TGN) to the plasma membrane. Other proteins have targeting signals for incorporation into specific organelles of the exocytic pathway, such as endosomes and lysosomes.

Lysosomes are acidic organelles in which endogenous and internalised macromolecules are degraded by luminal hydrolases. Endogenous macromolecules reach the lysosome by being sorted in the TGN from which they are transported to endosomes and then lysosomes.

The targeting signals used by a cell to sort proteins to the correct intracellular location may be exploited by the present invention. The signals may be broadly classed into the following types:

i) endocytosis signals

ii) Golgi retention signals

iii) TGN recycling signals

iv) ER retention signals

v) lysosomal sorting signals

The intracellular retention signal may direct the transmembrane protein away from the secretory pathway during translocation from the ER.

The intracellular retention signal may direct the transmembrane protein to an intracellular compartment or complex. The intracellular retention signal may direct the transmembrane protein to a membrane-bound intracellular compartment.

For example, the intracellular retention signal may direct the protein to a lysosomal, endosomal or Golgi compartment (trans-Golgi Network, ‘TGN’).

Within a normal cell, proteins arising from biogenesis or the endocytic pathway are sorted into the appropriate intracellular compartment following a sequential set of sorting decisions. At the plasma membrane, proteins can either remain at the cell surface or be internalised into endosomes. At the TGN, the choice is between going to the plasma membrane or being diverted to endosomes. In endosomes, proteins can either recycle to the plasma membrane or go to lysosomes. These decisions are governed by sorting signals on the proteins themselves.

Lysosomes are cellular organelles that contain acid hydrolase enzymes that break down waste materials and cellular debris. The membrane around a lysosome allows the digestive enzymes to work at the pH they require. Lysosomes fuse with autophagic vacuoles (phagosomes) and dispense their enzymes into the autophagic vacuoles, digesting their contents.

An endosome is a membrane-bounded compartment inside eukaryotic cells. It is a compartment of the endocytic membrane transport pathway from the plasma membrane to the lysosome and provides an environment for material to be sorted before it reaches the degradative lysosome. Endosomes may be classified as early endosomes, late endosomes, or recycling endosomes depending on the time it takes for endocytosed material to reach them. The intracellular retention signal may direct the protein to a late endosomal compartment.

The Golgi apparatus is part of the cellular endomembrane system, the Golgi apparatus packages proteins inside the cell before they are sent to their destination; it is particularly important in the processing of proteins for secretion.

There is a considerable body of knowledge which has arisen from studies investigating the sorting signals present in known proteins, and the effect of altering their sequence and/or position within the molecule (Bonifacino and Traub (2003) Ann. Rev. Biochem. 72:395-447; Braulke and Bonifacino (2009) Biochimica and Biophysica Acta 1793:605-614; Griffith (2001) Current Biology 11:R226-R228; Mellman and Nelson (2008) Nat Rev Mol Cell Biol. 9:833-845; Dell′Angelica and Payne (2001) Cell 106:395-398; Schafer et al (1995) EMBO J. 14:2424-2435; Trejo (2005) Mol. Pharmacol. 67:1388-1390). Numerous studies have shown that it is possible to insert one or more sorting signals into a protein of interest in order to alter the intracellular location of a protein of interest (Pelham (2000) Meth. Enzymol. 327:279-283).

Examples of endocytosis signals include those from the transferrin receptor and the asialoglycoprotein receptor.

Examples of signals which cause TGN-endosome recycling include those form proteins such as the Cl- and CD-MPRs, sortilin, the LDL-receptor related proteins LRP3 and LRP10 and β-secretase, GGA1-3, LIMP-II, NCP1, mucolipn-1, sialin, GLUT8 and invariant chain.

Examples of TGN retention signals include those from the following proteins which are localized to the TGN: the prohormone processing enzymes furin, PC7, CPD and PAM; the glycoprotein E of herpes virus 3 and TGN38.

Examples of ER retention signals include C-terminal signals such as KDEL (SEQ ID No. 9), KKXX (SEQ ID No. 10) or KXKXX (SEQ ID No. 11) and the RXR(R) (SEQ ID No. 12) motif of potassium channels. Known ER proteins include the adenovirus E19 protein and ERGIC53.

Examples of lysosomal sorting signals include those found in lysosomal membrane proteins, such as LAMP-1 and LAMP-2, CD63, CD68, endolyn, DC-LAMP, cystinosin, sugar phosphate exchanger 2 and acid phosphatase.

The molecule may comprise a Fas-binding domain coupled to an intracellular retention signal. Intracellular retention signals are well known in the art (see, for example, Bonifacino & Traub; Annu. Rev. Biochem.; 2003; 72; 395-447).

The term, “intracellular retention signal” refers to an amino acid sequence which directs or maintains the protein in which it is encompassed to a cellular compartment other than that to which it would be directed in the absence of the intracellular retention signal. In this case, the intracellular retention signal directs or maintains nascent Fas to a cellular compartment other than the cell surface membrane.

The intracellular retention signal may be any protein or protein domain which is a resident of a given intracellular compartment. This means that said protein or domain is in majority, located in a given compartment. At least 80%, 85%, 90%, 95%, 96%, 97%, 98% or 99% of said protein or domain is located in said compartment in a cell. The intracellular retention signal prevents the molecule of the present invention and any Fas molecule to which is it bound being translocated to the plasma membrane.

As used herein “compartment” or “subcellular compartment” refers to a given subdomain of cell. A compartment may be an organelle (such as endoplasmic reticulum, Golgi apparatus, endosome, lysosome) or an element of an organelle (such as multi-vesicular bodies of endosomes, cis-medial- or trans-cisternae of the Golgi apparatus etc.) or the plasma membrane or sub-domains of the plasma membrane (such as apical, basolateral, axonal domains) or micro domains such as focal adhesions or tight junctions.

An “intracellular compartment” refers to a compartment within a cell.

Endogenous Fas may be retained within the cell or within a specific intracellular compartment by the interaction with the Fas binding domain of the molecule of the invention which binding domain is coupled to an intracellular retention signal.

The intracellular retention signal may direct the protein to a Golgi (trans-Golgi Network, “TGN”), endosomal or lysosomal compartment.

The intracellular retention signal may be selected from the following group: a Golgi retention sequence; a trans-Golgi network (TGN) recycling signal; an endoplasmic reticulum (ER) retention sequence; a proteasome localization sequence or a lysosomal sorting signal.

The intracellular retention signal may be a protein or domain which is resident in the Golgi. Suitably, the Golgi retention domain may be selected from the group comprising: Giantin (GolgB1, GenBank Accession number NM+004487.3), TGN38/46, Menkes receptor and Golgi enzymes such as ManlI (α-1,3-1,6 mannosidase, Genbank accession number NM_008549), Sialyl Transferase (β-galactosamide α2,6-sialytransferae 1, NM_003032), GaIT (β-1,4-galactosyltransferase 1, NM_001497) adenoviral E19, HLA invariant chain or fragments thereof comprising the localisation domains.

The Golgi retention sequence comprises an amino acid sequence selected from: SEKDEL (SEQ ID NO: 13), KDEL (SEQ ID NO: 9), KKXX (SEQ ID NO: 10), KXKXX (SEQ ID NO: 11), a tail of adenoviral E19 protein comprising the sequence KYKSRRSFIDEKKMP (SEQ ID NO: 14), a fragment of HLA invariant chain comprising the sequence MHRRRSRSCR (SEQ ID NO: 15), KXD/E (SEQ ID NO: 16) or a YQRL (SEQ ID NO: 17) or variants thereof which retain the ability to function as Golgi retention sequences, wherein X is any amino acid.

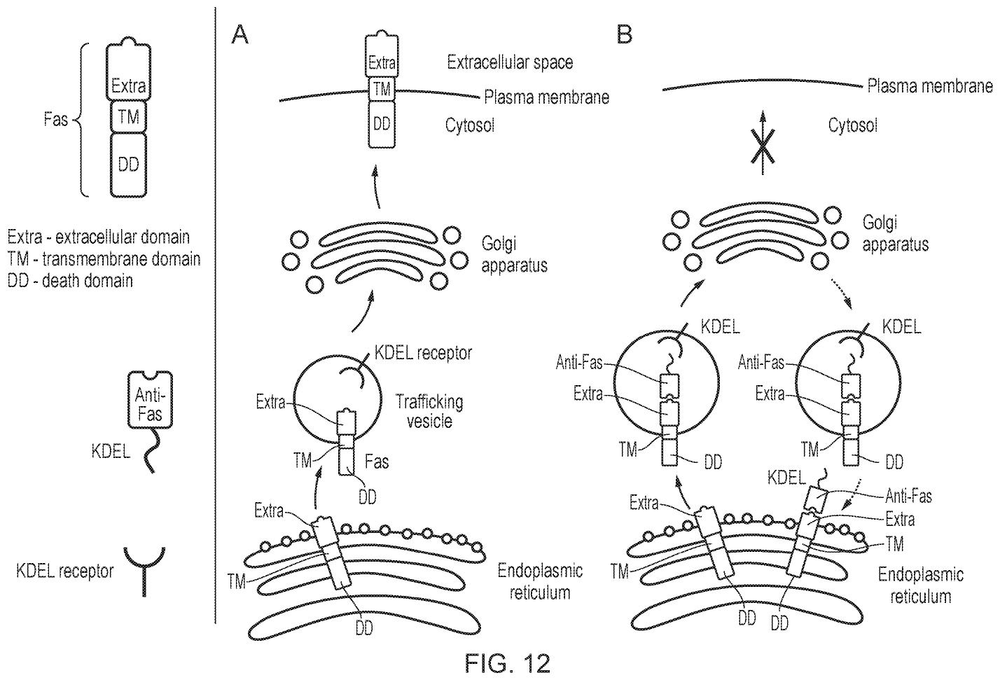

As mentioned above, the intracellular retention sequence may comprise a SEKDEL (SEQ ID NO: 13) or KDEL (SEQ ID NO: 9) sequence. For example, the KDEL sequence in the Fas-retaining molecules described in the Examples of the present application has the sequence DPAEPSEKDEL (SEQ ID No. 18). The KDEL receptor binds protein in the ER-Golgi intermediate compartment, or in the early Golgi and returns them to the ER. Proteins only leave the ER after the KDEL sequence has been cleaved off. Thus the protein resident in the ER will remain in the ER as long as it contains a KDEL sequence. This is illustrated schematically in FIG. 12.

The intracellular retention sequence may be located at the C-terminus of the molecule. Where the molecule of the invention is encoded by a nucleic acid sequence which is part of a bicistronic (or multi-cistronic) nucleic acid construct, it is preferable if the intracellular retention sequence encoding sequence is not located immediately upstream/5′ of a sequence encoding a self-cleaving peptide (such as a 2A or 2A-like peptide).

KKX′X′ and KX′KX′X′ signals are retrieval signals which can be placed on the cytoplasmic side of a type I membrane protein. Sequence requirements of these signals are provided in detail by Teasdale & Jackson (Annu. Rev. Cell Dev. Biol.; 12; 27 (1996)).

The retention signal may be a KKXX (SEQ ID NO: 10) motif. The KKXX domain may be located that the C terminus of the protein. KKXX is responsible for retrieval of ER membrane proteins from the cis end of the Golgi apparatus by retrograde transport, via interaction with the coat protein (COPI) complex.

The intracellular retention signal may be from the adenovirus E19 protein. The intracellular retention signal may be from the protein E3/19K, which is also known as E3gp 19 kDa; E19 or GP19K. The intracellular retention signal may comprise the full cytosolic tail of E3/19K; or the last 6 amino acids of this tail. Suitably, the retention signal may be a tail of adenoviral E19 protein comprising the sequence KYKSRRSFIDEKKMP (SEQ ID NO: 14). The retention signal may be a tail of adenoviral E19 protein comprising the sequence: DEKKMP (SEQ ID No. 19).

An ER retention signal may selected from the group comprising: an isoform of the invariant chain which resides in the ER (li33), Ribophorin I, Ribophorin II, SEC61 or cytochrome b5 or fragments thereof comprising the localisation domains. An example of an ER localisation domain is the ER localisation of Ribophorin II, Genbank accession BC060556.1.

The intracellular retention signal may be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

Tyrosine-based sorting signals mediate rapid internalization of transmembrane proteins from the plasma membrane and the targeting of proteins to lysosomes (Bonifacino & Traub; supra). Two types of tyrosine-based sorting signals are represented by the NPX′Y and YX′X′Z′ consensus motifs (wherein Z′ is an amino acid with a bulky hydrophobic side chain).

NPX′Y signals mediate rapid internalization of type I transmembrane proteins, they occur in families such as members of the LDL receptor, integrin 13, and β-amyloid precursor protein families.

Examples of NPX′Y signals are provided in Table 1.

TABLE 1
NPX′Y signals

| Protein | Species | Sequence |
|---|---|---|
| LDL receptor | Human | Tm-10-INFDNPVYQKTT-29 |
| LRP1 (1) | Human | Tm-21-VEIGNPTYKMYE-64 |
| LRP1 (2) | Human | Tm-55-TNFTNPVYATLY-33 |
| LRP1 | Drosophila | Tm-43-GNFANPVYESMY-38 |
| LRP1 (1) | C. elegans | Tm-54-TTFTNPVYELED-91 |
| LRP1 (2) | C. elegans | Tm-140-LRVDNPLYDPDS-4 |
| Megalin (1) | Human | Tm-70-IIFENPMYSARD- |
| | | 125 |
| Megalin (2) | Human | Tm-144-TNFENPIYAQME- |
| | | 53 |
| Integrin 13-1 (1) | Human | Tm-18-DTGENPIYKSAV-11 |
| Integrin 13-1 (2) | Human | Tm-30-TTVVNPKYEGK |
| Integrin 13 (1) | Drosophila | Tm-26-WDTENPIYKQAT-11 |
| Integrin 13 (2) | Drosophila | Tm-35-STFKNPMYAGK |
| APLP1 | Human | Tm-33-HGYENPTYRFLE-3 |
| APP | Human | Tm-32-NGYENPTYKFFE-4 |
| APP-like | Drosophila | Tm-38-NGYENPTYKYFE-3 |
| Insulin receptor | Human | Tm-36-YASSNPEYLSAS- |
| | | 379 |
| EGR receptor (1) | Human | Tm-434-GSVQNPVYHNQP- |
| | | 96 |
| EGR receptor (2) | Human | Tm-462-TAVGNPEYLNTV- |
| | | 68 |
| EGR receptor (3) | Human | Tm-496-ISLDNPDYQQDF-34 |

Numbers in parentheses indicate motifs that are present in more than one copy within the same protein. The signals in this and other tables should be considered examples. Key residues are indicated in bold type. Numbers of amino acids before (i.e., amino-terminal) and after (i.e., carboxy-terminal) the signals are indicated. Abbreviations: Tm, transmembrane; LDL, low density lipoprotein; LRP1, LDL receptor related protein 1; APP, 13-amyloid precursor protein; APLP1, APP-like protein 1.

YX′X′Z′-type signals are found in endocytic receptors such as the transferrin receptor and the asialoglycoprotein receptor, intracellular sorting receptors such as the Cl- and

CD-MPRs, lysosomal membrane proteins such as LAMP-1 and LAMP-2, and TGN proteins such as TGN38 and furin, as well as in proteins localized to specialized endosomal-lysosomal organelles such as antigen-processing compartments (e.g., HLA-DM) and cytotoxic granules (e.g., GMP-17). The YX′X′Z′-type signals are involved in the rapid internalization of proteins from the plasma membrane. However, their function is not limited to endocytosis, since the same motifs have been implicated in the targeting of transmembrane proteins to lysosomes and lysosome-related organelles.

Examples of YX′X′Z′-type signals are provided in Table 2.

TABLE 2
YX′X′Z′-type signals

| Protein | Species | Sequence |
|---|---|---|
| LAMP-1 | Human | Tm-RKRSHAGYQTI |
| LAMP-2a | Human | Tm-KHHHAGYEQF |
| LAMP-2a | Chicken | Tm-KKHHNTGYEQF |
| LAMP-2b | Chicken | Tm-RRKSRTGYQSV |
| LAMP-2C | Chicken | Tm-RRKSYAGYQTL |
| LAMP | Drosophila | Tm-RRRSTSRGYMSF |
| LAMP | Earthworm | Tm-RKRSRRGYESV |
| CD63 | Human | Tm-KSIRSGYEVM |
| GMP-17 | Human | Tm-HCGGPRPGYETL |
| GMP-17 | Mouse | Tm-HCRTRRAEYETL |
| CD68 | Human | Tm-RRRPSAYQAL |
| CD1b | Human | Tm-RRRSYQNIP |
| CD1c | Human | Tm-KKHCSYQDIL |
| CD1d | Mouse | Tm-RRRSAYQDIR |
| CD1 | Rat | Tm-RKRRRSYQDIM |
| Endolyn | Rat | Tm-KFCKSKERNYHTL |
| Endolyn | Drosophila | Tm-KFYKARNERNYHTL |
| TSC403 | Human | Tm-KIRLRCQSSGYQRI |
| TSC403 | Mouse | Tm-KIRQRHQSSAYQRI |
| Cystinosin | Human | Tm-HFCLYRKRPGYDQLN |
| Putative | Human | Tm-12-SLSRGSGYKEI |
| solute | | |
| carrier | | |
| TRP-2 | Human | Tm-RRLRKGYTPLMET-11 |
| HLA-DM [symbol] | Human | Tm-RRAGHSSYTPLPGS-9 |
| LmpA | Dictyosteliu | Tm-KKLRQQKQQGYQAIINNE |
| Putative | Dictyosteliu | Tm-RSKSNQNQSYNLIQL |
| lysosomal | | |
| LIMP-II | Dictyosteliu | Tm-RKTFYNNNQYNGYNIIN |
| Transferrin | Human | 16-PLSYTRFSLA-35-Tm |
| receptor | | |
| Asialo- | Human | MTKEYQDLQHL-29-Tm |
| glycoprotein | | |
| CI-MPR | Human | Tm-22-SYKYSKVNKE-132 |
| CD-MPR | Human | Tm-40-PAAYRGVGDD-16 |
| CTLA-4 | Human | Tm-10-TGVYVKMPPT-16 |
| Furin | Human | Tm-17-LISYKGLPPE-29 |
| TGN38 | Rat | Tm-23-ASDYQRLNLKL |
| gp41 | HIV-1 | Tm-13-RQGYSPLSFQT-144 |
| Acid | Human | Tm-RMQAQPPGYRHVADGEDHA |
| phosphatase | | |

Dileucine-based sorting signals ([DE]X′X′X′LL[LI]) play critical roles in the sorting of many type I, type II, and multispanning transmembrane proteins. Dileucine-based sorting signals are involved in rapid internalization and lysosomal degradation of transmembrane proteins and the targeting of proteins to the late endosomal-lysosomal compartments. Transmembrane proteins that contain constitutively active forms of this signal are mainly localised to the late endosomes and lysosomes.

Examples of [DE]X′X′X′LL[LI] sorting signals are provided in Table 3.

TABLE 3
[DE]X′X′X′LL[LI] sorting signals

| Protein | Species | Signal |
|---|---|---|
| CD3-γ | Human | Tm-8-SDKQTLLPN-26 |
| LIMP-II | Rat | Tm-11-DERAPLIRT |
| Nmb | Human | Tm-37-QEKDPLLKN-7 |
| QNR-71 | Quail | Tm-37-TERNPLLKS-5 |
| Pmel17 | Human | Tm-33-GENSPLLSG-3 |
| Tyrosinase | Human | Tm-8-EEKQPLLME-12 |
| Tyrosinase | Medaka fish | Tm-16-GERQPLLQS-13 |
| Tyrosinase | Chicken | Tm-8-PEIQPLLTE-13 |
| TRP-1 | Goldfish | Tm-7-EGRQPLLGD-15 |
| TRP-1 | Human | Tm-7-EANQPLLTD-20 |
| TRP-1 | Chicken | Tm-7-ELHQPLLTD-20 |
| TRP-2 | Zebrafish | Tm-5-REFEPLLNA-11 |
| VMAT2 | Human | Tm-6-EEKMAILMD-29 |
| VMAT1 | Human | Tm-6-EEKLAILSQ-32 |
| VAchT | Mouse | Tm-10-SERDVLLDE-42 |
| VAMP4 | Human | 19-SERRNLLED-88-Tm |
| Neonatal | Rat | Tm-16-DDSGDLLPG-19 |
| FcR | | |
| CD4 | Human | Tm-12-SQIKRLLSE-17 |
| CD4 | Cat | Tm-12-SHIKRLLSE-17 |
| GLUT4 | Mouse | Tm-17-RRTPSLLEQ-17 |
| GLUT4 | Human | Tm-17-HRTPSLLEQ-17 |
| IRAP | Rat | 46-EPRGSRLLVR-53-Tm |
| Ii | Human | MDDQRDLISNNEQLPMLGR-11- |
| | | Tm |
| Ii | Mouse | MDDQRDLISNHEQLPILGN-10- |
| | | Tm |
| Ii | Chicken | MAEEQRDLISSDGSSGVLPI-12- |
| | | Tm |
| Ii-1 | Zebrafish | MEPDHQNESLIQRVPSAETILGR- |
| | | 12-Tm |
| Ii-2 | Zebrafish | MSSEGNETPLISDQSSVNMGPQP- |
| | | 8-Tm |
| Lamp | Trypanosome | Tm-RPRRRTEEDELLPEEAEGLID |
| | | PQN |
| Menkes | Human | Tm-74-PDKHSLLVGDFREDDD |
| protein | | TAL |
| NPC1 | Human | Tm-13-TERERLLNF |
| AQP4 | Human | Tm-32-VETDDLIL-29 |
| RME-2 | C. elegans | Tm-104-FENDSLL |
| Vam3p | S. cerevisiae | 153-NEQSPLLHN-121-Tm |
| ALP | S. cerevisiae | 7-SEQTRLVP-18-Tm |
| Gap1p | S. cerevisiae | Tm-23-EVDLDLLK-24 |

DX′X′LL signals constitute a distinct type of dileucine-based sorting signals. These signals are present in several transmembrane receptors and other proteins that cycle between the TGN and endosomes, such as the Cl- and CD-MPRs, sortilin, the LDL-receptor-related proteins LRP3 and LRP10, and β-secretase.

Examples of DX′X′LL sorting signals are provided in Table 4.

TABLE 4
DX′X′LL sorting signals

| Protein | Species | Sequence |
|---|---|---|
| CI-MPR | Human | Tm-151-SFHDDSDEDLLHI |
| CI-MPR | Bovine | Tm-150-TFHDDSDEDLLHV |
| CI-MPR | Rabbit | Tm-151-SFHDDSDEDLLNI |
| CI-MPR | Chicken | Tm-148-SFHDDSDEDLLNV |
| CD-MPR | Human | Tm-54-EESEERDDHLLPM |
| CD-MPR | Chicken | Tm-54-DESEERDDHLLPM |
| Sortilin | Human | Tm-41-GYHDDSDEDLLE |
| SorLA | Human | Tm-41-ITGFSDDVPMVIA |
| Head-activator | Hydra | Tm-41-INRFSDDEPLVVA |
| BP | | |
| LRP3 | Human | Tm-237-MLEASDDEALLVC |
| ST7 | Human | Tm-330-KNETSDDEALLLC |
| LRP10 | Mouse | Tm-235-WVVEAEDEPLLA |
| LRP10 | Human | Tm-237-WVAEAEDEPLLT |
| Beta-secretase | Human | Tm-9-HDDFADDISLLK |
| Mucolipin-1 | Mouse | Tm-43-GRDSPEDHSLLVN |
| Nonclassical | Deer mouse | Tm-6-VRCHPEDDRLLG |
| MHC-I | | |
| FLJ30532 | Human | Tm-83-HRVSQDDLDLLTS |
| GGA1 | Human | 350-ASVSLLDDELMSL-275 |
| GGA1 | Human | 415-ASSGLDDLDLLGK-211 |
| GGA2 | Human | 408-VQNPSADRNLLDL-192 |
| GGA3 | Human | 384-NALSWLDEELLCL-326 |
| GGA | Drosophila | 447-TVDSIDDVPLLSD-116 |

Another family of sorting motifs is provided by clusters of acidic residues containing sites for phosphorylation by CKII. This type of motif is often found in transmembrane proteins that are localized to the TGN at steady state, including the prohormone-processing enzymes furin, PC6B, PC7, CPD, and PAM, and the glycoprotein E of herpes virus 3.

Examples of acidic cluster signals are provided in Table 5.

TABLE 5
Acidic cluster sorting signals

| Protein | Species | Sequence |
|---|---|---|
| Furin | Mouse | Tm-31-QEECPSDSEEDEG-14 |
| PC6B (1)^{a} | Mouse | Tm-39-RDRDYDEDDEDDI-36 |
| PC6B (2) | Mouse | Tm-69- |
| | Human | LDETEDDELEYDDES-4 |
| PC7 | Human | Tm-38-KDPDEVETES-47 |
| | Human | |
| CPD PAM | Human | Tm-36-HEFQDETDTEEET-6 |
| | Human | |
| VMAT2 | Human HCMV | Tm-59-QEKEDDGSESEEEY-12 |
| VMAT1 | | Tm-35-GEDEESESD |
| VAMP4 | | Tm-35-GEDSDEEPDHEE |
| Glycoprotein | Herpes | Tm-28-FEDSESTDTEEEF-21 |
| | virus 3 | |
| E Nef | HIV-1 | 55-LEAQEEEEV-139 |
| | (AAL65476) | |
| Kex1p (1) | S. | Tm-29- |
| | cerevisiae | ADDLESGLGAEDDLEQDEQLEG-40 |
| Kex1p (2) | S. | Tm-79-TEIDESFEMTDF |
| | cerevisiae | |
| Kex2p | S. | Tm-36- |
| | cerevisiae | TEPEEVEDFDFDLSDEDH-61 |
| Vps10p | S. | Tm-112-FEIEEDDVPTLEEEH-37 |
| | cerevisiae | |

The intracellular retention signal may be selected from the group of: NPX′Y, YX′X′Z, [DE]X′X′X′L[LI], DX′X′LL, DP[FVV], FX′DX′F, NPF, LZX′Z[DE], LLDLL, PWDLW, KDEL, HDEL, KKX′X′ or KX′KX′X′; wherein X′ is any amino acid and Z′ is an amino acid with a bulky hydrophobic side chain.

The intracellular retention signal may be any sequence shown in Tables 1 to 5.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 20) or a variant thereof.

TYRP1 is a well-characterized melansomal protein which is retained in the melanosome (a specialized lysosome) at >99% efficiency. TYRP1 is a 537 amino acid transmembrane protein with a lumenal domain (1-477aa), a transmembrane domain (478-501), and a cytoplasmic domain (502-537). A di-leucine signal residing on the cytoplasmic domain causes retention of the protein. This di-leucine signal has the sequence shown as SEQ ID No. 20 (NQPLLTD).

Linker

The molecule may comprise a linker to connect the Fas-binding domain to the intracellular retention signal. The linker may, for example, be a peptide linker. Numerous suitable peptide linker sequences are known in the art and/or could be readily designed by a skilled person.

Flexible polypeptide linkers composed of glycine and serine are commonly used to connect separate domains of engineered multidomain proteins. Such a linker may, for example, comprise repeats of the sequence (GGGGS)n (SEQ ID NO: 21) or, in which n is an integer of, e.g., about 1 to about 8. The linker may comprise the sequence GGGGSGGGGS (SEQ ID NO: 22) or SGGGSGGGSGGGS (SEQ ID NO: 23). The anti-Fas KDEL molecule having the amino acid sequence shown in FIG. 8 has a linker with the sequence shown as SED ID No. 23.

The peptide linker used in the molecule may have a length of about 5 to about 40; about 10 to about 30; or about 10 to about 20 amino acids.

Signal Peptide

The molecule may comprise a signal peptide so that when it is expressed inside a cell, the nascent protein is directed to the endoplasmic reticulum. The signal peptide may, for example, be an IL-2 signal peptide, a kappa leader sequence, a CD8 leader sequence or a peptide with essentially equivalent activity.

The construct shown in FIG. 8 encodes an anti-Fas KDEL molecule with an Ig Kappa leader sequence having the following sequence MGWSCIILFLVATATGVHS (SEQ ID No. 24).

The molecule invention may have the sequence shown as SEQ ID No. 25 which is made up of an mlg Kappa Leader sequence (signal peptide), the light chain from the anti-Fas antibody F45D9 (shown above as SEQ ID No. 6), a GS linker, the heavy chain from the anti-Fas antibody F45D9 (shown above as SEQ ID No. 5) and the intracellular retention sequence shown above as SEQ ID No. 18.

(anti-Fas-KDEL)
SEQ ID No. 25
MGWSCIILFLVATATGVHSDIQMTQSPSSLSASVGDRVTITCRASQGIRR
WLAWYQQKPEKAPKSLIYAASSLQSGVPSRFSGSGSGTDFTLTISSLQPE
DFATYYCQQYNSYPYTFGQGTKLEIKRSGGGGSGGGGSGGGGSQVQLQQW
GAGLLKPSETLSLICAVYGGSFSTYYWTWIRQPPGKGLEWIGEINHRGTT
NYSPSLKSRVTISVDTSKNHISLNLTSVTAADTALYYCARGLLWIGEGDY
GLDVWGQGTTVTVSSDPAEPSEKDEL

Dominant Negative Fas

The present invention provides a vector expressing a nucleic acid sequence which inhibits Fas expression in the cell. The nucleic acid sequence may, for example, encode a dominant negative Fas molecule.

A dominant negative Fas polypeptide acts antagonistically to the wild-type allele, i.e. it adversely affects the normal, wild-type Fas polypeptide within the same cell. A dominant negative Fas polypeptide may interact with a wild-type Fas polypeptide, but blocks its signal transduction to downstream molecules, e.g., FADD.

A dominant negative Fas polypeptide may comprise at least one modification in a cytoplasmic death domain of human Fas. The modification may be selected from the group consisting of mutations, deletions, and insertions. For example, the mutation may be deletion or a point mutation.

The modification may be in the cytoplasmic death domain of human Fas. It may prevent the binding between the dominant negative Fas polypeptide and a FADD polypeptide. In other words, the dominant negative Fas polypeptide may comprise the Fas extracellular domain but has a truncated or mutated death domain so that it does not bind FADD.

The dominant negative Fas molecule may lack the Fas endodomain or may lack the FADD-binding domain. Where the Fas endodomain is completely absent, it may be replaced by an alternative endodomain, such as an endodomain from another molecule. The replacement endodomain may be “inert” in the sense that it functions to stabilise the molecule in terms of trans-membrane expression but does not confer any additional properties on the molecule. Alternatively, the replacement endodomain may confer one or more additional properties to the molecule such as the capacity to induce or co-stimulate T-cell signalling.

A truncated Fas endodomain may have a deletion in a central portion of the Fas endodomain. For example, it may comprise a deletion of about 50, 60, 70, 80 or 90 amino acids. The deletion may include the portion of sequence from residues 240-290, 235-295 or 230-300 of a human Fas having the amino acid sequence set forth in SEQ ID NO: 1.

WO2020/069508 describes a dominant negative Fas polypeptide which comprises a deletion of amino acids 230-314 of a human Fas having the amino acid sequence set forth in SEQ ID NO: 1.

In the examples of the present application, a dominant negative Fas is used lacking amino acid residues 222-306 of the sequence shown as SEQ ID No. 1. A truncated Fas endodomain which lacking this portion of the sequence is shown below as SEQ ID No. 26. This sequence corresponds to SEQ ID No. 4, with the underlined portion of the sequence removed.

(truncated Fas endodomain)
SEQ ID No. 26
KRKEVQKTCRKHRKENQGSHESPTLNPETVAAEKIQTIILKDITSDSENS
NFRNEIQSLV

In the respect, the dominant negative Fas molecule may comprise the Fas ectodomain (SEQ ID No. 2) and a truncated version of Fas endodomain having the sequence shown as SEQ ID No. 26. The dominant negative Fas molecule may, for example have the sequence shown as SEQ ID No. 27.

(truncated Fas, or FasΔDD)
SEQ ID No. 27
QVTDINSKGLELRKTVTTVETQNLEGLHHDGQFCHKPCPPGERKARDCTV
NGDEPDCVPCQEGKEYTDKAHFSSKCRRCRLCDEGHGLEVEINCTRTQNT
KCRCKPNFFCNSTVCEHCDPCTKCEHGIIKECTLTSNTKCKEEGSRSNLG
WLCLLLLPIPLIVWVKRKEVQKTCRKHRKENQGSHESPTLNPETVAAEKI
QTIILKDITSDSENSNFRNEIQSLV

The cell of the present invention may comprise a truncated version of Fas which comprises a deletion of amino acids 230-314 or 222-306 of a human Fas having the amino acid sequence set forth in SEQ ID NO: 1; a truncated version of Fas which comprises the sequence shown as SEQ ID No. 27 or a polypeptide which is at least 80, 90, 95 or 99% identical to such a sequence provided that the resultant dominant negative Fas molecule competes with endogenous Fas for binding to FasL and has no or reduced capacity to bind FADD.

The dominant negative Fas molecule may alternatively comprise on or more point mutations which reduce or abolish its capacity to bind FADD. WO2020/069508 describes a dominant negative Fas polypeptide which comprises a point mutation at position 260 of a human Fas having the amino acid sequence set forth in SEQ ID NO: 1. The cell of the present invention may comprise a mutated version of Fas which comprises such a point mutation. The point mutation may be, for example, be D260V. This sequence is shown below as SEQ ID No. 28.

(Fas with D260V point mutation)
SEQ ID No. 28
MLGIWTLLPLVLTSVARLSSKSWAQVTDINSKGLELRKTVTTVETQNLEG
LHHDGQFCHKPCPPGERKARDCTWGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLSKYITTIAGVMTLSQVKGFVRKN
GWEAKIVEIKNDNVQDTAEQKVQLLRNWHQLHGKKEAYDTLIKDLKKANL
CTLAEKIQTIILKDITSDSENSNFRNEIQSLV

The dominant negative Fas molecule may comprise a signal peptide so that it is exported to the cell surface. The signal peptide may be derived from Fas or it may be a heterologous signal peptide (i.e. a signal peptide derived from another protein or a synthetic signal peptide).

A dominant negative Fas molecule may comprise the Fas extracellular domain and a heterologous endodomain, i.e. an endodomain derived from another protein. For example the dominant negative Fas molecule may comprise the Fas extracellular domain and a endodomain from a TNF receptor. Such a molecule may comprise the Fas transmembrane domain (SEQ ID No. 3), or any other suitable transmembrane sequence.

A summary of TNFRs and their ligands is provided in Table 6.

TABLE 6

| Protein | | | |
|---|---|---|---|
| (member #) | Synonyms | Gene | Ligand(s) |
| Tumor necrosis | CD120a | TNFRSF1A | TNF-alpha |
| factor receptor 1 | | | (cachectin) |
| Tumor necrosis | CD120b | TNFRSF1B | TNF-alpha |
| factor receptor 2 | | | (cachectin) |
| Lymphotoxin | CD18 | LTBR | Lymphotoxin |
| beta receptor | | | beta (TNF-C) |
| OX40 | CD134 | TNFRSF4 | OX40L |
| CD40 | Bp50 | CD40 | CD154 |
| Fas receptor | Apo-1, CD95 | FAS | FasL |
| Decoy receptor 3 | TR6, M68 | TNFRSF6B | FasL, LIGHT, |
| | | | TL1A |
| CD27 | S152, Tp55 | CD27 | CD70, Siva |
| CD30 | Ki-1 | TNFRSF8 | CD153 |
| 4-1BB | CD137 | TNFRSF9 | 4-1BB ligand |
| Death receptor 4 | TRAILR1, Apo-2, | TNFRSF10A | TRAIL |
| | CD261 | | |
| Death receptor 5 | TRAILR2, CD262 | TNFRSF10B | TRAIL |
| Decoy receptor 1 | TRAILR3, LIT, | TNFRSF10C | TRAIL |
| | TRID, CD263 | | |
| Decoy receptor 2 | TRAILR4, | TNFRSF10D | TRAIL |
| | TRUNDD, | | |
| | CD264 | | |
| RANK | CD265 | TNFRSF11A | RANKL |
| Osteoprotegerin | OCIF, TR1 | TNFRSF11B | |
| TWEAK receptor | Fn14, CD266 | TNFRSF12A | TWEAK |
| TACl | IGAD2, CD267 | TNFRSF13B | APRIL, |
| | | | BAFF, |
| | | | CAMLG |
| BAFF | CD268 | TNFRSF13C | BAFF |
| receptor | | | |
| Herpesvirus | ATAR, TR2, | TNFRSF14 | LIGHT |
| entry | CD270 | | |
| mediator | | | |
| Nerve growth | p75NTR, CD271 | NGFR | NGF, BDNF, |
| factor receptor | | | NT-3, NT-4 |
| B-cell | TNFRSF13A, | TNFRSF17 | BAFF |
| maturation | CD269 | | |
| antigen | | | |
| Glucocorticoid- | AITR, CD357 | TNFRSF18 | GITR ligand |
| induced TNFR- | | | |
| related | | | |
| TROY | TAJ, TRADE | TNFRSF19 | unknown |
| Death receptor 6 | CD358 | TNFRSF21 | |
| Death receptor 3 | Apo-3, TRAMP, | TNFRSF25 | TL1A |
| | LARD, WS-1 | | |
| Ectodysplasin | XEDAR | EDA2R | EDA-A2 |
| A2 receptor | | | |

The TNFR-derived endodomain may be derived from any of the TNFRs listed in Table 6. For example the endodomain may be derived from decoy receptor 2 (DcR2), GITR, CD30, XEDAR, CD40, CD27, BCMA, Fn14 or 41BB. The sequences for these endodomains are shown below.

(DcR2 endodomain)
SEQ ID No. 29
RKKFISYLKGICSGGGGGPERVHRVLFRRRSCPSRVPGAEDNARNETLSN
RYLQPTQVSEQEIQGQELAELTGVTVESPEEPQRLLEQAEAEGCQRRRLL
VPVNDADSADISTLLDASATLEEGHAKETIQDQLVGSEKLFYEEDEAGSA
TSCL
(GITR endodomain)
SEQ ID No. 30
QLGLHIWQLRSQCMWPRETQLLLEVPPSTEDARSCQFPEEERGERSAEEK
GRLGDLWV
(CD30 endodomain)
SEQ ID No. 31
HRRACRKRIRQKLHLCYPVQTSQPKLELVDSRPRRSSTQLRSGASVTEPV
AEERGLMSQPLMETCHSVGAAYLESLPLQDASPAGGPSSPRDLPEPRVST
EHTNNKIEKIYIMKADTVIVGTVKAELPEGRGLAGPAEPELEEELEADHT
PHYPEQETEPPLGSCSDVMLSVEEEGKEDPLPTAASGK
(XEDAR endodomain)
SEQ ID No. 32
LYCKQFFNRHCQRGGLLQFEADKTAKEESLFPVPPSKETSAESQVSENIF
QTQPLNPILEDDCSSTSGFPTQESFTMASCTSESHSHWVHSPIECTELDL
QKFSSSASYTGAETLGGNTVESTGDRLELNVPFEVPSP
(CD40 endodomain)
SEQ ID No. 33
KKVAKKPTNKAPHPKQEPQEINFPDDLPGSNTAAPVQETLHGCQPVTQED
GKESRISVQERQ
(CD27 endodomain)
SEQ ID NO. 34
RKQRRKYRSNKGESPVEPAEPCHYSCPREEEGSTIPIQEDYRKPEPACSP
(BCMA endodomain)
SEQ ID No. 35
RKINSEPLKDEFKNTGSGLLGMANIDLEKSRTGDEIILPRGLEYTVEECT
CEDCIKSKPKVDSDHCFPLPAMEEGATILVTTKTNDYCKSLPAALSATEI
EKSISAR
(Fn14 endodomain)
SEQ ID No. 36
RRCRRREKFTTPIEETGGEGCPAVALIQ
(41BB endodomain)
SEQ ID No. 37
KRGRKKLLYIFKQPFMRPVQTTQEEDGCSCRFPEEEEGGCEL

WO2018/170475 describes a fusion protein comprising a Fas ectodomain and a 4-1BB endodomain.

Fas Knockout/Knockdown

The cell of the present invention may comprise one or more nucleic acid sequence(s) which inhibits Fas expression in the cell. The nucleic acid sequence(s) may reduce expression of the Fas gene (knockdown) or disrupt the Fas gene completely (knockout).

There are various methods known in the art for knocking out or knocking down the expression of a protein in a cell. Genome editing techniques include using Zinc finger nucleases (ZFNs), transcription activator-like effector nucleases (TALENs), small interfering RNA (siRNA), short hairpin RNA (shRNA) and clustered regularly interspaced short palindromic repeat (CRISPR) technology.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. The cell of the invention may comprise a nucleic acid sequence encoding a Fas-specific ZFN to knockout expression of the Fas gene.

TALENs are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. The cell of the invention may express Fas-specific pair of TALENs to knockout expression of the Fas gene.

siRNA

Small interfering RNA (siRNA) is a class of double-stranded RNA non-coding RNA molecules, typically 20-27 base pairs in length, similar to miRNA, and operating within the RNA interference (RNAi) pathway. It interferes with the expression of specific genes with complementary nucleotide sequences by degrading mRNA after transcription, preventing translation.

Naturally occurring siRNAs have a well-defined structure that is a short (usually 20 to 24-bp) double-stranded RNA (dsRNA) with phosphorylated 5′ ends and hydroxylated 3′ ends with two overhanging nucleotides. The Dicer enzyme catalyzes production of siRNAs from long dsRNAs and small hairpin RNAs.

An expression vector may be used to express siRNA within cells. The siRNA sequence is modified to introduce a short loop between the two strands. The resulting transcript is a short hairpin RNA (shRNA), which can be processed into a functional siRNA by Dicer in its usual fashion. Typical transcription cassettes use an RNA polymerase III promoter (e.g., U6 or H1) to direct the transcription of small nuclear RNAs (snRNAs).

Brummelkamp et al (2002, Science 296:550-553) describes a vector system, pSUPER, which directs the synthesis of siRNAs in mammalian cells. The polymerase III Hi-RNA gene promoter is cloned in front of the gene specific targeting sequence: a short nucleotide sequence from the target transcript separated by a short spacer from the reverse complement of the same sequence, and five thymidines as termination signal.

Retroviral constructs have now been developed in which the polymerase-Ill H1-RNA gene promoter synthesizes siRNA-like transcripts allowing stable expression of siRNA. Dotti et al (2005, Blood 105:4677-4684) describe the use of retroviral siRNA directed against Fas mRNA to knock down this molecule in human Epstein-Barr virus-specific cytotoxic T lymphocytes (EBV-CTLs). Their data show that stable knock down of Fas can be achieved in EBV-CTLs by transduction with retroviral siRNA and that modified cells became resistant to Fas/FasL-mediated apoptosis, allowing selection of the Fasb^{low} population.

In this study, pSUPER.puro and pSUPER.GFP vectors were used to make retroviral vectors containing various different sequences predicted as siRNA for Fas mRNA. Two of the sequences tested: GGACATTACTAGTGACTCA (SEQ ID No. 38) and GTTCAACTGCTTCGTAATT (SEQ ID No. 39) substantially knocked down Fas expression.

The cell of the present invention may comprise an siRNA sequence which knocks down Fas expression. The cell may comprise an siRNA sequence which targets a Fas target recognition sequence. For example, the cell may comprise an siRNA sequence which targets the Fas target recognition sequence shown as SEQ ID No. 38 or 39.

The vector of the present invention may encode an siRNA sequence which knocks down Fas expression. For example, it may encode the sense siRNA sequence 5′-pGUGCAAGUGCAAACCAGACdTdT-3′ (SEQ ID No. 40) and the antisense siRNA 5′-pGUCUGGUUUGCACUUGCACdTdT-3′ (SEQ ID No. 41)

shRNA

A short hairpin RNA or small hairpin RNA (shRNA/Hairpin Vector) is an artificial RNA molecule with a tight hairpin turn that can be used to silence target gene expression via RNA interference (RNAi). A variety of viral vectors can be used to obtain shRNA expression in cells including adeno-associated viruses (AAVs), adenoviruses, and lentiviruses. Once the vector has integrated into the host genome, the shRNA is then transcribed in the nucleus by polymerase II or polymerase III depending on the promoter choice. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing.

The cell of the present invention may comprise an shRNA sequence which knocks down Fas expression. The vector of the invention may comprise a nucleic acid sequence encoding an shRNA which targets a sequence on the Fas gene. The vector of the invention may comprise the sequence shown as SEQ ID No. 42 which encodes an anti-Fas shRNA sequence.

(Fas shRNA)
SEQ ID No. 42
CCGGGTGCAGATGTAAACCAAACTTCTCGAGAAGTTTGGTTTACATCTG
CACTTTTTG

CRISPR

The CRISPR (Clustered regularly interspaced short palindromic repeats)-Cas system is a prokaryotic immune system that confers resistance to foreign genetic elements, such as those present within plasmids and phages, and provides a form of acquired immunity.

Cas9 (or “CRISPR-associated protein 9”) is an enzyme that uses CRISPR sequences as a guide to recognize and cleave specific strands of DNA that are complementary to the CRISPR sequence. Cas9 enzymes, together with CRISPR sequences, form the basis of a technology known as CRISPR-Cas9 that can be used to edit genes within organisms. This editing process has a wide variety of applications including basic biological research, development of biotechnology products, and treatment of diseases.

CRISPR/Cas9 is a target-specific technique that can introduce gene knock out or knock in depending on the double strand repair pathway. The targeting specificity of CRISPR-Cas9 is determined by the 20-nt sequence at the 5′ end of the guide RNA (gRNA). The desired target sequence must precede the protospacer adjacent motif (PAM) which is a short DNA sequence usually 2-6 base pairs in length that follows the DNA region targeted for cleavage by CRISPR-Cas9. The PAM is required for a Cas nuclease to cut and is generally found 3-4 nucleotides downstream from the cut site. After base pairing of the gRNA to the target, Cas9 mediates a double strand break about 3-nt upstream of PAM.

CRISPR-Cas9 enables multiplex genome editing by the expression of multiple gRNAs. Ren et al ((2017) Oncotarget 8(10):17002-17011) describe a one-shot CRISPR protocol for multiplex gene editing of CAR-T cells by incorporating multiple gRNA expression cassettes in a single CAR lentiviral vector. Using this technique, they describe the double knockout of endogenous T-cell receptor (TCR) and HLA class I (HLA-I) (described in more detail below). They also describe Fas-resistant CAR-T cells created by gene disruption of the Fas gene. Triple gene editing using the one-shot CRISPR system was achieved by expressing a TRAC gRNA under a human U6 promoter, a TRBC gRNA under mouse U6 promoter and a Fas gRNA expressed under a third promoter selected from: sU6, 7SL, 7SK, h5S, m5S, H1.

The Fas-targeting gRNA-encoding sequence used in that study is shown below as SEQ ID No. 43

(Fas-gRNA encoding sequence)
SEQ ID No. 43
GAGGGTCCAGATGCCCAGCA

The cell of the invention may comprise a Fas-targeting gRNA sequence encoded by SEQ ID No. 43 or it may comprise one of the Fas-targeting gRNA sequences shown below as SEQ ID No. 44 to 47. The vector of the invention may encode one of the Fas-targeting gRNA sequences shown below as SEQ ID No. 36 to 39.

SEQ ID No. 44
GGAGUUGAUGUCAGUCACUU
SEQ ID No. 45
GUGACUGACAUCAACUCCAA
SEQ ID No. 46
CUUCCUCAAUUCCAAUCCCU
SEQ ID No. 47
UGACAUCAACUCCAAGGGAU

INHIBITING Fas ACTIVITY

The cell of the present invention may comprise one or more nucleic acid sequence(s) which inhibits Fas activity in the cell. The nucleic acid sequence may encode a molecule which binds to and/or competitively inhibits Fas. The nucleic acid sequence may encode a molecule which competes with Fas for binding to FasL. The molecule may be membrane-bound DcR3.

DcR3

DcR3 is a soluble receptor that has no signal transduction capabilities (hence a “decoy”) and functions in vivo to prevent FasR-FasL interactions by competitively binding to soluble and membrane-bound Fas ligand.

The cell of the invention may express a membrane-bound DcR3 molecule.

The amino acid sequence for DcR3 is available from Uniport (Accession No. 095407). In that sequence amino acids 1-29 form the signal peptide. The DcR3 sequence without the signal peptide is shown below as SEQ ID No. 57.

(human DcR3 without signal peptide)
SEQ ID No. 57
VAETPTYPWRDAETGERLVCAQCPPGTFVQRPCRRDSPTTCGPCPPRHY
TQFWNYLERCRYCNVLCGEREEEARACHATHNRACRCRTGFFAHAGFCL
EHASCPPGAGVIAPGTPSQNTQCQPCPPGTFSASSSSSEQCQPHRNCTA
LGLALNVPGSSSHDTLCTSCTGFPLSTRVPGAEECERAVIDFVAFQDIS
IKRLQRLLQALEAPEGWGPTPRAGRAALQLKLRRRLTELLGAQDGALLV
RLLQALRVARMPGLERSVRERFLPVH

The structure of DcR3 comprises four N-terminal cysteine-rich domains (CRD) that bind FasL, and a C-terminal heparan-binding domain (HBD) that binds to heparan sulphate proteoglycans (HSPG). It has been shown that the HBD of DcR3, but not the FasL-binding CRDs, induce apoptosis in dendritic cells via cross-linking of HSPGs (You et al., 2008, Blood 111, 1480-1488).

The HBD of DcR3 binds to HSPGs via a binding motif comprising three basic amino acids (K²⁵⁶, R²⁵⁸ and R²⁵⁹), and mutating these to alanines abolishes binding to HSPGs.

(mutant DcR3)
SEQ ID NO: 58
VAETPTYPWRDAETGERLVCAQCPPGTFVQRPCRRDSPTTCGPCPPRHY
TQFWNLERCRYCNVLCGEREEEARACHATHNRACRCRTGFFAHAGFCLE
HASCPPGAGVIAPGTPSQNTQCQPCPPGTFSASSSSSEQCQPHRNCTAL
GLALNVPGSSSHDTLCTSCTGFPLSTRVPGAEECERAVIDFVAFQDISI
KRLQRLLQALEAPEGWGPTPRAGRAALQLALAARLTELLGAQDGALLVR
LLQALRVARMPGLERSVRERFLPVH

The cell of the invention may express a membrane-bound form of DcR3 in which the extracellular domain comprises the sequence shown as SEQ ID No. 52 or 53 or a variant thereof having 80, 85, 90, 95 or 99% identity to the sequence shown as SEQ ID No. 57 or 58, provided that the variant sequence retains the capacity to bind FasL.

The membrane-bound DcR3 molecule may comprise a spacer sequence in order to distance DcR3 from the membrane and provide flexibility. Spacers described below which are commonly used in CARs such as: an IgG1 Fc domain; an IgG1 hinge; an IgG1 hinge-CD8 stalk; or a CD8 stalk may also be used in the membrane-bound DcR3 molecule. A table of sequences (SEQ ID NO's: 59 to 65) suitable for tethering Dc3R to the plasma membrane is provided herein (Table 7). A membrane proximal KPDK buffer sequence may be added to these spacers.

TABLE 7
Sequences suitable for tethering DcR3 to the plasma membrane

| SEQ IDs | Amino acid sequence |
|---|---|
| SEQ ID NO: | PTTTPAPRPPTPAPTIASQPLSLRPEACRPAAGGAVHTRGLDFACD |
| 59 (CD8stk) | |
| SEQ ID NO: | EPKSCDKTHTCPPCP |
| 60 (Hinge) | |
| SEQ ID NO: | AGSDLGPQMLRELQETNAALQDVRELLRQQVREITFLKNTVMECDACGSG |
| 61 (COMP) | K |
| SEQ ID NO: | KGPSVFPLAPSSKSTSGGTAALGCLVKDYFPEPVTVSWNSGALTSGVHTFP |
| 62 (CH1- | AVLQ |
| Hinge) | SSGLYSLSSVVTVPSSSLGTQTYICNVNHKPSNTKVDKRVEPKSCDKTHTC |
| | PPCP |
| SEQ ID NO: | TKGPSVFPLAPSSKSTSGGTAALGCLVKDYFPEPVTVSWNSGALTSGVHTF |
| 63 (CH1- | PAVLQ |
| Hinge-CH2- | SSGLYSLSSVVTVPSSSLGTQTYICNVNHKPSNTKVDKRVEPKSCDKTHTC |
| CH3) | PPCPA |
| | PPVAGPSVFLFPPKPKDTLMIARTPEVTCVVVDVSHEDPEVKFNWYVDGVE |
| | VHNAK |
| | TKPREEQYNSTYRVVSVLTVLHQDWLNGKEYKCKVSNKALPAPIEKTISKAK |
| | GQPRE |
| | PQVYTLPPSRDELTKNQVSLTCLVKGFYPSDIAVEWESNGQPENNYKTTPP |
| | VLDSDG |
| | SFFLYSKLTVDKSRWQQGNVFSCSVMHEALHNHYTQKSLSLSPGK |
| SEQ ID NO: | RTVAAPSVFIFPPSDEQLKSGTASVVCLLNNFYPREAKVQWKVDNALQSGN |
| 64 (HuIG | SQES |
| Kappa) | VTEQDSKDSTYSLSSTLTLSKADYEKHKVYACEVTHQGLSSPVTKSFNRGE |
| | C |
| SEQ ID NO: | AKIEVMYPPPYLDNEKSNGTIIHVKGKHLCPSPLFPGPSKP |
| 65 (CD28 | |
| Stalk) | |

The membrane-bound DcR3 may comprise any one of the spacer sequences shown as SEQ ID NOs: 59 to 65 or a variant thereof. Variants may be of an equivalent length and/or have at least 80, 85, 90, 95, 98 or 99% sequence identity to SEQ ID NOs: 59 to 65.

The membrane-bound DcR3 molecule may include a sequence linking the DcR3 sequence to the spacer, such as for example, a standard three-amino acid SDP linker sequence.

The membrane-bound DcR3 molecule may include a transmembrane sequence to anchor the DcR3 sequence into the membrane. The transmembrane domain may be any protein structure which is thermodynamically stable in a membrane e.g. does not dimerize. This is typically an alpha helix comprising of several hydrophobic residues. The transmembrane domain of any transmembrane protein can be used to supply a transmembrane portion. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e., a polypeptide sequence predicted to form a hydrophobic alpha helix of sufficient length to span the membrane, an artificially designed TM domain may also be used (U.S. Pat. No. 7,052,906 B1 describes transmembrane components).

Suitable transmembrane domains may be derived from, for example, CD8a, CD28, DcR2, TYRP-1 or EGFR.

The membrane-bound DcR3 molecule may include a polar anchor after the transmembrane domain, to provide a polar region in close proximity to the plasma membrane.

(Polar anchor)
SEQ ID NO: 66
RKKR

The membrane-bound DcR3 molecule may comprise an endodomain. The endodomain may be “inert” in the sense that it functions to stabilise the molecule in terms of trans-membrane expression but does not confer any additional properties on the molecule. Alternatively, the endodomain may confer one or more additional properties to the molecule such as the capacity to induce or co-stimulate T-cell signalling.

An example of an inert endodomain is a rigid linker amino acid sequence which is commonly used to covalently join functional domains. This sequence is less flexible that the commonly used Glycine-Serine linker and is advantageous at the C-terminal end of a recombinant protein sequence. A suitable rigid linker sequence is shown as SEQ ID No. 67

(Rigid linker with truncations)
SEQ ID NO: 67
LEAEAAAKEAAAKEAAAKEAAAKALEAEAAAKEAAAKEAAAKEAAAKALE

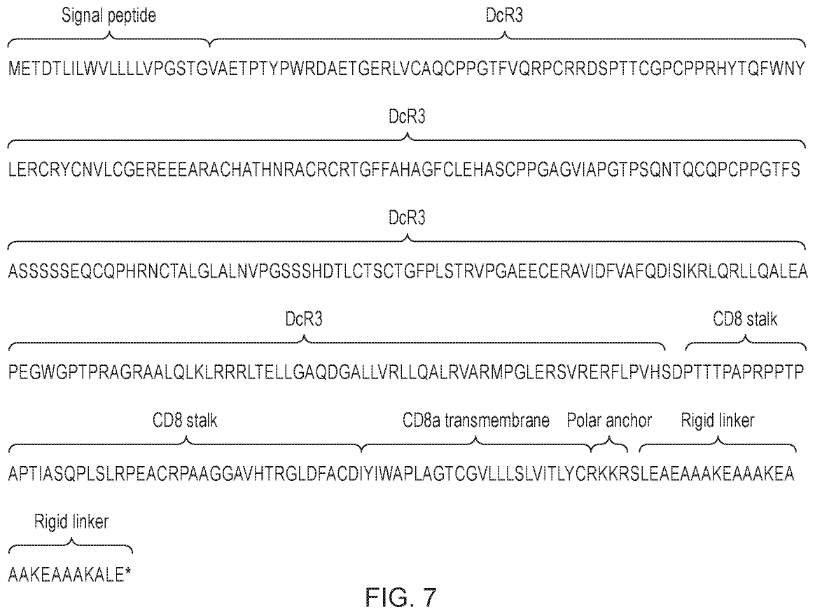

The annotated sequence for a full membrane-bound DcR3 sequence having the format: Human DcR3-CD8 stalk/TM/rigid linker is shown in FIG. 7. This sequence has a spacer sequence and transmembrane domain derived from CD8a.

The membrane-bound DcR3 molecule may comprise a stimulatory endodomain, such as a CD3 endodomain. The amino acid sequence for CD3z endodomain is shown as SEQ ID No. 48.

The membrane-bound DcR3 molecule may comprise a stimulatory endodomain such as an endodomain selected from one of the following proteins: CD28, ICOS, CTLA4, 41BB, CD27, CD30, OX-40, TACl, GITR, CD2 and CD40. The amino acid sequences for these endodomains are shown above.

Fas LIGAND

Fas ligand (FasL or CD95L or CD178) is a type-II transmembrane protein that belongs to the tumor necrosis factor (TNF) family. FasL is a homotrimeric type II transmembrane protein expressed on cytotoxic T lymphocytes. As explained above and illustrated schematically in FIG. 9, Fas binding to FasL triggers trimerization of Fas, resulting in apoptosis of the Fas-expressing cell.

The present invention provides a method for selecting transduced cells based on their capacity to resist apoptosis following exposure to FasL.

In the method of the invention, FasL may be soluble, bound to a solid substrate such as a bead or plate, or expressed on the surface of a cell.

Soluble Fas ligand may generated by cleaving membrane-bound FasL at a conserved cleavage site by the external matrix metalloproteinase MMP-7. It may trimerized or multimerised by cross-linking. For example hexameric proteins containing two trimers of FasL within the same molecule may be made by fusing FasL to the Fc portion of immunoglobulin G1 or to the collagen domain of ACRP30/adiponectin.

FasL has two receptors: Fas (as described above) and Decoy receptor 3 (DcR3), a recently discovered decoy receptor of the tumor necrosis factor superfamily. DcR3 is a soluble receptor that has no signal transduction capabilities (hence a “decoy”) and functions to prevent FasR-FasL interactions by competitively binding to membrane-bound or soluble Fas ligand and rendering them inactive.

FasL has the sequence shown as SEQ ID No. 7 above. Of this sequence, amino acids 1-80 are the cytoplasmic domain; 81-102 are the transmembrane domain; and amino acids 103-281 are the extracellular domain. The amino acid sequence of the FasL extracellular domain alone is shown above as SEQ ID No. 8.

As mentioned above, in the method of the invention, FasL may be expressed on the surface of a cell. In this respect, the method of the invention may involve co-culturing the cell of the invention with a cell which expresses FasL. The cell may naturally express FasL or may be engineered to express or over-express FasL. In order to select transduced cells using the method of the invention, the cell only needs to express the portion of FasL which binds Fas, i.e. the FasL extracellular domain. The FasL expressing cell may therefore be engineered to express an alternative protein, such as a fusion protein which comprises the FasL extracellular domain and a heterologous endodomain.

The present invention also provides cells which cell having reduced expression or activity of Fas which co-express an NOI and FasL.

The NOI may, for example, inhibit expression of endogenous T-cell receptor (TCR) or encode a CAR or a transgenic T cell receptor TCR.

The cell may express FasL having the sequence shown as SEQ ID No. 7 or a variant thereof having 80, 85, 90, 95 or 99% identity to the sequence shown as SEQ ID No. 7, provided that the variant sequence retains the capacity to bind Fas.

The cell may express as FasL fusion protein which comprises the Fas-binding domain of FasL together with a heterologous portion. For example, the cell may express a protein comprising the FasL ectodomain with a heterologous endodomain. The FasL extracellular domain may have the sequence shown as SEQ ID No. 8 or a variant thereof having 80, 85, 90, 95 or 99% identity to the sequence shown as SEQ ID No. 8, provided that the variant sequence retains the capacity to bind Fas.

The heterologous endodomain may be derived from another protein, such as a protein involved in T cell signalling or co-stimulation.

The FasL fusion protein may, for example comprise the endodomain from CD3z which is shown below as SEQ ID No. 48.

(human CD3G endodomain)
SEQ ID No. 48
SRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTY
DALHMQALPPR

A co-stimulatory endodomain may be or comprise an endodomain selected from one of the following proteins: CD28 (SEQ ID No. 49), ICOS (SEQ ID No. 50), CTLA4 (SEQ ID No. 51), 41BB (SEQ ID No. 37), CD27 (SEQ ID No. 34), CD30 (SEQ ID No. 31), OX-40 (SEQ ID No. 52), TACl (SEQ ID No. 53), GITR (SEQ ID No. 30), CD2 (SEQ ID No. 54) and CD40 (SEQ ID No. 33).

(CD28 endodomain)
SEQ ID No. 49
RSKRSRLLHSDYMNMTPRRPGPTRKHYQPYAPPRDFAAYRS
(ICOS endodomain)
SEQ ID No. 50
CWLTKKKYSSSVHDPNGEYMFMRAVNTAKKSRLTDVTL
(CTLA4 endodomain)
SEQ ID No. 51
AVSLSKMLKKRSPLTTGVYVKMPPTEPECEKQFQPYFIPIN
(OX-40 endodomain)
SEQ ID No. 52
ALYLLRRDQRLPPDAHKPPGGGSFRTPIQEEQADAHSTLAKI
(TACI endodomain)
SEQ ID No. 53
KKRGDPCSCQPRSRPRQSPAKSSQDHAMEAGSPVSTSPEPVETCSFCFP
ECRAPTQESAVTPGTPDPTCAGRWGCHTRTTVLQPCPHIPDSGLGIVCV
PAQEGGPGA
(CD2 endodomain)
SEQ ID No. 54
KRKKQRSRRNDEELETRAHRVATEERGRKPHQIPASTPQNPATSQHPPP
PPGHRSQAPSHRPPPPGHRVQHQPQKRPPAPSGTQVHQQKGPPLPRPRV
QPKPPHGAAENSLSPSS

The fusion protein may comprise a combination of endodomains, such as CD28 and CD3z or 4-1BB and CD3z.

The fusion protein may be selected from one of the following:
FasL-CD3z
FasL-CD28z
FasL-41BBz

Non-Cleavable Fas Ligand

The cell of the invention may express a mutated version of FasL which is non-cleavable. Fas ligand, like its family members TNF-α and TRAIL, is cleaved by metalloproteases from a 40-kDa membrane-anchored form to a soluble, 26-29-kDa form. The use of a non-cleavable version makes FasL more effective in inducing Fas-mediated apoptosis.

The cleavage sites of FasL is between residues Ser-126 and Leu-127 which reside in the extracellular domain. Sang-Mo et al (2000, Transplantation 69:1813-1817) describe deletion mutants created to generate a non-cleavable FasL. They showed FasL containing deletions of residues 126-135 and 126-145 were more potent than WT FasL in inducing apoptosis.

The cell of the invention may express mutant FasL comprising a deletion of residues 126-135 or 126-145 of the sequence shown as SEQ ID No. 7. These mutant FasL molecules have the sequence shown as SEQ ID No. 55 and 56 below.

(mutant FasL having a deletion of residues 126-135)
SEQ ID No. 55
MQQPFNYPYPQIYWWDSSASSPWAPPGTVLPCPTSVPRRPGQRRPPPPP
PPPPLPPPPPPPPLPPLPLPPLKKRGNHSTGLCLLVMFFMVLVALVGLG
LGMFQLFHLQKELAELRESTSQMHTASPPPEKKELRKVAHLTGKSNSRS
MPLEWEDTYGIVLLSGVKYKKGGLVINETGLYFVYSKVYFRGQSCNNLP
LSHKVYMRNSKYPQDLVMMEGKMMSYCTTGQMWARSSYLGAVFNLTSAD
HLYVNVSELSLVNFEESQTFFGLYKL
(mutant FasL having a deletion of residues 126-145)
SEQ ID No. 56
MQQPFNYPYPQIYWWDSSASSPWAPPGTVLPCPTSVPRRPGQRRPPPPP
PPPPLPPPPPPPPLPPLPLPPLKKRGNHSTGLCLLVMFFMVLVALVGLG
LGMFQLFHLQKELAELRESTSQMHTASVAHLTGKSNSRSMPLEWEDTYG
IVLLSGVKYKKGGLVINETGLYFVYSKVYFRGQSCNNLPLSHKVYMRNS
KYPQDLVMMEGKMMSYCTTGQMWARSSYLGAVFNLTSADHLYVNVSELS
LVNFEESQTFFGLYKL

Nucleic Acid of Interest

The present invention provides a method for selecting for cells transduced to express a nucleic acid sequence of interest (NOI), which comprises the following steps:
(a) transducing a population of cells with a vector co-expressing the NOI and a nucleic acid sequence which inhibits Fas expression in the cell;
(b) exposing the cells from (a) to FasL such that untransduced cells are eliminated by apoptosis.

In a first embodiment of this aspect of the invention, the NOI encodes a protein of interest (POI). The POI may be any recombinant polypeptide expressed in a cell.

For example, the NOI may encode a chimeric antigen receptor (CAR) or a transgenic T cell receptor (TCR).

CAR

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain may be used to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ξ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3 results in second generation receptors which can transmit an activating and co-stimulatory signal simultaneously after antigen recognition. The co-stimulatory domain most commonly used is that of CD28. This supplies the most potent co-stimulatory signal —namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain—spacer domain—transmembrane domain—intracellular signaling domain (endodomain).

Antigen Binding Domain

The antigen binding domain is the portion of the CAR which recognizes antigen. In a classical CAR, the antigen-binding domain comprises: a single-chain variable fragment (scFv) derived from a monoclonal. CARs have also been produced with domain antibody (dAb), VHH or Fab-based antigen binding domains.

Alternatively a CAR may comprise a ligand for the target antigen. For example, B-cell maturation antigen (BCMA)-binding CARs have been described which have an antigen binding domain based on the ligand a proliferation inducing ligand (APRIL).

Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

A variety of sequences are commonly used as spacers for CAR, for example, an IgG1 Fc region, an IgG1 hinge, or a human CD8 stalk.

Transmembrane Domain

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:

(i) an ITAM-containing endodomain, such as the endodomain from CD3 zeta; and/or

(ii) a co-stimulatory domain, such as the endodomain from CD28 or ICOS; and/or

(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the present invention may therefore comprise an antigen-binding component comprising an antigen-binding domain and a transmembrane domain; which is capable of interacting with a separate intracellular signalling component comprising a signalling domain. The vector of the invention may express a CAR signalling system comprising such an antigen-binding component and intracellular signalling component.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

Target Antigen

A ‘target antigen’ is an entity which is specifically recognised and bound by the antigen-binding domain of a CAR.

The target antigen may be an antigen present on a cancer cell, for example a tumour-associated antigen.

Various tumour associated antigens (TAA) are known, as shown in the following Table 8. The CAR may be capable of binding such a TAA.

TABLE 8

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Transgenic T-Cell Receptor

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (a) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vector. For example the genes for engineered TCRs may be reintroduced into autologous T cells and transferred back into patients for T cell adoptive therapies. Such ‘heterologous’ TCRs may also be referred to herein as ‘transgenic TCRs’.

Inhibition of Tcr Expression

In a second embodiment of the method of the invention, the NOI inhibits expression of endogenous T-cell receptor (TCR). The NOI may cause knockout of the endogenous TCR gene, knockdown of TCR gene expression or reduce the cell-surface expression of the endogenous TCR. For example, the NOI may encode:
(i) a TCR or CD3-binding domain linked to an intracellular retention signal;
(ii) a gRNA molecule comprising a targeting domain that is complementary with a target domain in a TCR gene; or
(iii) an siRNA complementary with a target domain in a TCR gene.

(i) a TCR or CD3-binding domain linked to an intracellular retention signal;

TCR Retention

The cell of the present invention may express a molecule which comprises a TCR or CD3-binding domain linked to an intracellular retention signal. Suitable molecules are described in US2018/0086831 which is herein incorporated by reference.

TCR Knockout/Knockdown

The cell of the present invention may comprise one or more nucleic acid sequence(s) which inhibits TCR expression in the cell. The NOI(s) may reduce expression of a TCR gene (knockdown) or disrupt the an endogenous TCR gene completely (knockout). The NOI may inhibit expression of a TRA, TRB, TRG and/or TRD gene.

Alternatively the cell of the invention may comprise one or more nucleic acid sequence(s) which inhibits CD3 expression in the cell. Targeting of the CD3 complex causes impaired TCR assembly and reduced TCR cell surface expression. The approach may, for example, target CD3e.

As explained above, there are various methods known in the art for knocking out or knocking down the expression of a protein in a cell, including using Zinc finger nucleases (ZFNs), transcription activator-like effector nucleases (TALENs), small interfering RNA (siRNA), short hairpin RNA (shRNA) and clustered regularly interspaced short palindromic repeat (CRISPR) technology.

The NOI may encode a TCR- or CD3-specific ZFN or a TCR- or CD3-specific pair of TALENs to knockout expression of an endogenous TCR or CD3 gene.

The NOI may encode an siRNA or shRNA complementary with a target domain in a TCR or CD3 gene so that TCR or CD3 mRNA gets degraded. Okamoto et al (2012 Mol Ther Nucleic Acids 1(12) e63) describe siRNAs to knockdown endogenous TCR genes. Raitano et al (2018 DOI: 10.1200/JCO.2018.36.15_suppl.e15040 Journal of Clinical Oncology 36, no. 15_suppl) describe shRNA targeting of the CD3 complex.

The NOI may encode a gRNA molecule comprising a targeting domain that is complementary with a target domain in a TCR or CD3 gene. Ren et al (2017, as above) describe CRISPR/Cas-9 knockout of the endogenous TCR gene using a gRNA targeting TCR α chain constant region (TRAC) or targeting the TCR β chain constant region (TRBC). The gRNAs targeted the following sequences.

(TRAC-gRNA)
SEQ ID No. 68
AGAGTCTCTCAGCTGGTACA
(TRBC-gRNA)
SEQ ID No. 69
GCAGTATCTGGAGTCATTGA

Preventing Immune Rejection

WO2019/073248 describes a strategy to engineer therapeutic T cells to eliminate alloreactive T-cells in vivo and in vitro which involves linking β2-microglobulin (β2m), a universal component of all class I molecules, to a signalling domain, such as the cytolytic domain of CD3. This molecule forms a complex with endogenous HLA-class I alpha chains within the cell. Subsequently, when an alloreactive T cell binds to such and HLA complex on the therapeutic cell, the signalling domain is activated causing elimination of the alloreactive T cell. WO2020/208346 describes a similar strategy for deleting alloreactive MHC class II cells

In this respect, the cell of the invention may co-expresses β2-microglobulin (β2m), an MHC class I or MHC class II polypeptide, CD79 or CD4 linked to a signalling domain and/or a costimulatory domain.

Human β2-microglobulin (β2m) has the sequence shown as SEQ ID No. 70

(Human 32m)
SEQ ID No. 70
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM

A β2m polypeptide sequence for use in the present invention may comprise the sequence shown as SEQ ID NO: 70 or a variant thereof having at least 80% sequence identity. A variant of SEQ ID NO: 70 may have at least 80, 85, 90, 95, 98 or 99% sequence identity, provided that the variant sequence retains the capacity to assemble with a MHC class I protein and facilitate productive peptide presentation by the MHC class I complex.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

Endogenous β2m polypeptides do not comprise a transmembrane domain. The molecule of the cell of the invention a transmembrane domain located between the β2m polypeptide and the endodomain comprising an intracellular signalling domain.

The transmembrane domain may be any peptide domain that is capable of inserting into and spanning the cell membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components). For example, the transmembrane domain may comprise a hydrophobic alpha helix. The transmembrane domain may, for example, be derived from CD8alpha or CD28.

The intracellular signalling domain or co-stimulatory domain may, for example, by the endodomain from CD3z, CD28, 41BB or OX40. The sequences for the CD3z endodomain is shown above as SEQ ID No. 48. A co-stimulatory endodomain may be or comprise an endodomain selected from one of the following proteins: CD28, ICOS, CTLA4, 41BB, CD27, CD30, OX-40, TACT, GITR, CD2 and CD40. The amino acid sequences for these endodomains are shown above.

The fusion protein may comprise a combination of endodomains, such as CD28 and OX-40 or CD28 and 4-1BB.

The cell of the present invention may comprise and/or the nucleic acid construct of the invention may encode the following combination of β2m and FasL fusion proteins:
β2m-CD3z+FasL-CD28
β2m-CD3z+FasL-41 BB
β2m-CD28+FasL-CD3z
β2m-41 BB+FasL-CD3z

Preventing Target Cell Fight Back

GB patent application No. 2005216.3 describes various approaches for engineering an effector immune cell (cell A) such that, when targeting an autoreactive or pathogenic immune cell (cell B), the engineered immune cell has a selective advantage and the balance between the cell A killing cell B; and cell B killing cell A is tipped in favour of cell A killing cell B.

The effector immune cell is engineered such that when a synapse is formed between the effector immune cell and the target immune cell, the capacity of the effector immune cell to kill the target immune cell is greater than the capacity of the target immune cell to kill the effector immune cell.

This may be achieved for example, by engineered the effector immune cell to be resistant to the effector immune cell is engineered to be resistant to an immunosuppressant, such as a calcineurin inhibitors.

The effector cell of the present invention may comprise one or more mutations which increases its resistance to one or more immune suppressive drugs. For example, effector cell may comprise one or more mutations which renders the cell resistant to tacrolimus and/or cyclosporin.

The effector cell may comprise a nucleic acid sequence encoding calcineurin (CN) with one or more mutations. Calcineurin (CaN) is a calcium and calmodulin dependent serine/threonine protein phosphatase which activates the T cells of the immune system. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), stimulating the T cell response. Calcineurin is the target of a class of drugs called calcineurin inhibitors, which include cyclosporin, voclosporin, pimecrolimus and tacrolimus. Brewin et al (2009; Blood 114: 4792-4803) describe various calcineurin mutants which render cytotoxic T lymphocytes resistant to tacrolimus and/or cyclosporin.

Calcineurin is a heterodimer of a 61-kD calmodulin-binding catalytic subunit, calcineurin A and a 19-kD Ca2+-binding regulatory subunit, calcineurin B. There are three isozymes of the catalytic subunit, each encoded by a separate gene (PPP3CA, PPP3CB, and PPP3CC) and two isoforms of the regulatory, also encoded by separate genes (PPP3R1, PPP3R2). The amino acid sequences for all of the polypeptides encoded by these genes are available from Uniprot, with the following accession numbers: PPP3CA: Q08209; PPP3CB: P16298; PPP3CC: P48454; PPP3R1: P63098; and PPP3R2: Q96LZ3.

The amino acid sequence for calcineurin A, alpha isoform is shown below as SEQ ID No. 71

(calcineurin A)
SEQ ID No. 71
MSEPKAIDPKLSTTDRVVKAVPFPPSHRLTAKEVFDNDGKPRVDILKAH
LMKEGRLEESVALRIITEGASILRQEKNLLDIDAPVTVCGDIHGQFFDL
MKLFEVGGSPANTRYLFLGDYVDRGYFSIECVLYLWALKILYPKTLFLL
RGNHECRHLTEYFTFKQECKIKYSERVYDACMDAFDCLPLAALMNQQFL
CVHGGLSPEINTLDDIRKLDRFKEPPAYGPMCDILWSDPLEDFGNEKTQ
EHFTHNTVRGCSYFYSYPAVCEFLQHNNLLSILRAHEAQDAGYRMYRKS
QTTGFPSLITIFSAPNYLDVYNNKAAVLKYENNVMNIRQFNCSPHPYWL
PNFMDVFTWSLPFVGEKVTEMLVNVLNICSDDELGSEEDGFDGATAAAR
KEVIRNKIRAIGKMARVFSVLREESESVLTLKGLTPTGMLPSGVLSGGK
QTLQSATVEAIEADEAIKGFSPQHKITSFEEAKGLDRINERMPPRRDAM
PSDANLNSINKALTSETNGTDSNGSNSSNIQ

Mutant calcineurin A may comprise a mutation at one or more of the following positions with reference to SEQ ID No. 71: V314; Y341; M347; T351; W352; S353; L354; F356; and K360.

Mutant calcineurin A may comprise one or more of the following substitution mutations with reference to SEQ ID No. 71:
V314K, V314R or V314F;
Y341F;
M347W, M347R or M347E;
T351E;
W352A, W3520 or W352E;
S353H or S353N;
L354A;
F356A; and
K360A or K360F.

Mutant calcineurin A may comprise one or more of the following mutation combinations with reference to SEQ ID No. 71:
L354A and K360A;
L354A and K360F;
T351E and K360F;
W352A and S353H;
T351E and L354A;
W3520 and K360F;
W3520; L354A and K360F;
V314K and Y341F; and
V314R and Y341F.

The amino acid sequence for calcineurin B, type 1 is shown below as SEQ ID No. 72

(calcineurin B)
SEQ ID No. 72
MGNEASYPLEMCSHFDADEIKRLGKRFKKLDLDNSGSLSVEEFMSLPELQ
QNPLVQRVIDIFDTDGNGEVDFKEFIEGVSQFSVKGDKEQKLRFAFRIYD
MDKDGYISNGELFQVLKMMVGNNLKDTQLQQIVDKTIINADKDGDGRISF
EEFCAVVGGLDIHKKMVVDV

Mutant calcineurin B may comprise a mutation at one or more of the following positions with reference to SEQ ID No. 72: Q51; L116; M119; V120; G121; N122; N123; L124; K125; and K165.

Mutant calcineurin B may comprise one or more of the following substitution and optionally insertion mutations with reference to SEQ ID No. 72:
Q51S;
L116R or L116Y;
M119A, M119W or M119-F-Ins;
V120L, V1205, V120D or V120F;
G121-LF-Ins;
N122A, N122H, N122F or N1225;
N123H, N123R, N123F, N123K or N123W;
L124T;
K125A, K125E, K125W, K125-LA-Ins, K125-VQ-Ins or K125-IE-Ins; and
K165Q.

Mutant calcineurin B may comprise one or more of the following mutation
combinations with reference to SEQ ID No. 72:
V1205 and L124T;
V120D and L124T;
N123W and K125-LA-Ins;
L124T and K125-LA-Ins;
V120D and K125-LA-Ins; and
M119-F-Ins and G121-LF-Ins.

In the study described in Brewin et al 2009 (as above), the following CNa mutants showed resistance to FK506:
L354A and K360F;
W352A;
W3520;
T351E and L354A;
M347W; and
M347E.

The following CNa mutants showed resistance to cyclosporin A:
V314K;
V314R;
Y341F;
V314K and Y341F; and
V314R and Y341F.

The following CNb mutants showed resistance to FK506:
N123W;
K125-VQ-Ins;
K125-1E-Ins;
K-125-LA-Ins; and
L124T and K-125-LA-Ins.

The following CNb mutants showed resistance to cyclosporin A:
K125-VQ-Ins;
K125-1E-Ins;
K-125-LA-Ins;
V120S and L124T; and
L124T and K-125-LA-Ins.

In particular, it is reported in Brewin et al 2009 (as above) that:
the combination mutation T351E and L354A in CNa confers resistance to CsA but not FK506;
the combination mutation V314R and Y341F in CNa confers resistance to FK506 but not CsA; and
the combination mutation L124T and K-125-LA-Ins in CNb renders CTLs resistant to both calcineurin inhibitors.

The effector immune cell of the present invention may express a variant calcineurin A comprising one or more mutations in the CNa amino acid sequence and/or a variant calcineurin B comprising one or more mutations in the CNb amino acid sequence, which increases resistance of the effector immune cell to one or more calcineurin inhibitors.

In particular, the effector immune cell may express a variant calcineurin A and/or a variant calcineurin B as listed above which confers resistance to cyclosporin A and/or tacrolimus (FK506).

Nucleic Acid Construct

The present invention provides a nucleic acid construct which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR); and
a nucleic acid sequence encoding one of the following:
   (i) dominant negative Fas;
   (ii) Fas-binding domain linked to an intracellular retention signal;
   (iii) a gRNA molecule comprising a targeting domain that is complementary with a target domain in the Fas gene; or
   (iv) an siRNA complementary with a target domain in the Fas gene.

The present invention also provides a nucleic acid construct which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a nucleic acid sequence encoding FasL; and
a nucleic acid sequence encoding one of the following:
   (i) dominant negative Fas;
   (ii) Fas-binding domain linked to an intracellular retention signal;
   (iii) a gRNA molecule comprising a targeting domain that is complementary with a target domain in the Fas gene; or
   (iv) an siRNA or shRNA complementary with a target domain in the Fas gene.

The nucleic acids may be in any order in the construct.

Where the nucleic acid construct encodes shRNA, siRNA or a gRNA this element may be under the control of a U6 promoter. U6 is a type III RNA polymerase III promoter commonly used for driving shRNA or siRNA expression in vector-based RNAi and for driving gRNA expression.

Nucleic acids encoding two or more polypeptides may be separated by a co-expression site enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No.73 (RAEGRGSLLTCGDVEENPGP).

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Vector

The present invention also provides a vector, or kit of vectors, which comprises one or more nucleic acid sequence(s) or nucleic acid construct(s) according to the invention. Such a vector may be used to introduce the nucleic acid sequence(s) into a host cell so that it expresses one or more of the following proteins and/or NOI(s):
(i) an NOI or protein which inhibits expression of Fas, which may be selected from:
   (a) a molecule which comprises a Fas-binding domain linked to an intracellular retention signal;
   (b) dominant negative Fas
   (c) a gRNA molecule comprising a targeting domain that is complementary with a target domain in the Fas gene; or
   (d) an siRNA complementary with a target domain in the Fas gene; and/or
(ii) chimeric antigen receptor (CAR) or a transgenic T cell receptor (TCR); and/or
(iii) an NOI or protein which inhibits expression of endogenous T-cell receptor (TCR), which may be selected from:
   (a) a TCR or CD3-binding domain linked to an intracellular retention signal;
   (b) a gRNA molecule comprising a targeting domain that is complementary with a target domain in a TCR gene; or
   (c) an siRNA complementary with a target domain in a TCR gene; and/or
(iv) FasL or a FasL fusion protein; and/or
(v) β2m fused to a signalling and/or co-stimulatory domain; and/or
(vi) membrane-bound DcR3 or a fusion protein comprising DcR3; and/or
(vii) mutant calcineurin A and/or calcineurin B.

The invention provides a vector which comprises a nucleic acid construct for expression in a cell which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a nucleic acid sequence which inhibits Fas expression or activity in the cell; and optionally
a nucleic acid sequence encoding FasL

The vector may, for example, be a plasmid or a viral vector, such as a retroviral vector or a lentiviral vector, or a transposon based vector or synthetic mRNA.

The vector may be capable of transfecting or transducing an immune effector cell, such as a T cell or a NK cell.

The method of the present invention involves transducing a cell population with a vector. The vector co-expresses an NOI and a nucleic acid sequence which inhibits Fas expression in the cell.

Kit of Nucleic Acid Sequences/Vectors

The invention also provides a kit of nucleic acid sequences which comprises:
(i) a nucleic acid construct comprising: a nucleic acid sequence encoding a chimeric antigen receptor (CAR) or a transgenic T cell receptor (TCR) and/or a nucleic acid sequence which inhibits expression of endogenous T-cell receptor (TCR); and a nucleic acid sequence which inhibits Fas expression or activity in the cell; and
(ii) a nucleic acid sequence encoding FasL

The invention also provides a kit of vectors which comprises:
a first vector comprising a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR); and a nucleic acid sequence which inhibits Fas expression or activity when expressed in a cell; and
a second vector comprising a nucleic acid sequence encoding FasL

The present invention also provides a kit of vectors which comprises:
(a) a first vector which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR); and
a nucleic acid sequence encoding one of the following:
   (i) dominant negative Fas;
   (ii) Fas-binding domain linked to an intracellular retention signal;
   (iii) a gRNA molecule comprising a targeting domain that is complementary with a target domain in the Fas gene; or
   (iv) an siRNA complementary with a target domain in the Fas gene; and
(b) a second vector which comprises a nucleic acid sequence encoding FasL.

The kit of vectors may be in the form of a composition for co-transduction.

Selection Method

The invention provides a method for selecting for cells transduced to express a nucleic acid sequence of interest (NOI), which comprises the following steps:
(a) transducing a population of cells with a vector co-expressing (i) the NOI and (ii) a nucleic acid sequence which inhibits Fas expression in the cell;
(b) exposing the cells from (a) to FasL.

Cells transduced with the vector are resistant to FasL because they comprise the nucleic acid sequence which inhibits Fas expression in the cell. Untransduced cells, on the other hand, are eliminated by FasL-mediated apoptosis.

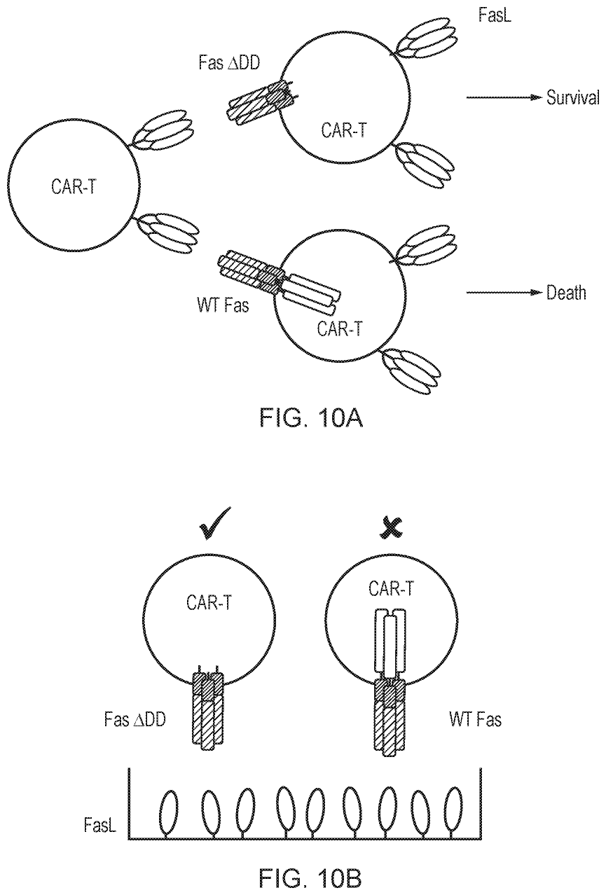

This process using an immobilised FasL is illustrated schematically in FIG. 10B

Cells may be exposed to soluble FasL, FasL bound to a solid substrate such as a plate or bead, or FasL expressed on the surface of a cell.

Cells may be exposed to FasL in its natural trimeric form or in higher order, multimerised form, such as hexamers. Hexameric preparations of FasL, such as MegaFasL™ are commercially available.

The invention also provides a method comprising the following steps:
(a) transducing a population of cells with a plurality of vectors, one of which co-expresses the NOI and a nucleic acid sequence which inhibits Fas expression in the cell;
(b) exposing the cells from (a) to FasL.

Because cells expressing the NOI have resistance to Fas L, cells transduced with the vector expressing the NOI or cells transduced with a combination of vectors including the vector expressing the NOI are spared from apoptosis, whereas untransduced cells or cells transduced with one or more vector(s) other than the vector expressing the NOI will be eliminated by apoptosis.

Self-Selection

The invention also provides a method which comprises the following steps:
(a) transducing a population of cells with a vector co-expressing (i) an NOI, (ii) a nucleic acid sequence which inhibits Fas expression in the cell and (iii) a nucleic acid sequence encoding FasL;
(b) culturing the cells.

Due to the expression of FasL on the surface of transduced cells, untransduced cells (with normal levels of Fas expression) will be eliminated by apoptosis; whereas transduced cells (in which Fas expression is reduced or eliminated) will be spared.

Culturing the transduced cell population therefore has the effect of “self-selecting” for cells transduced with the vector. This is illustrated schematically in FIG. 10A.

There is also provided a method which comprises the following steps:
(a) co-transducing a population of cells with:
   a first vector expressing (i) a CAR or TCR; and (ii) means for inhibiting Fas expression in the cell; and
   a second vector expressing FasL
(b) culturing the cells.

This method has the effect or selecting for cells transduced with the first vector, together with cells co-transduced with the first and second vector. Cells transduced with the second vector alone will die due to Fas ligation.

The second vector may co-express FasL and a second polypeptide, such as an “enhancement module” which has an effect on the CAR- or TCR-expressing cell.

An “enhancement module” may, for example, be a constitutively active cytokine receptor, such as those described in WO2017/029512; or a cytokine, such as IL-12.

Following a period of culture, cells expressing the enhancement module without the CAR or engineered TCR will be eliminated by FasL-mediated apoptosis, so the resultant cell population will be a mixture of cells which express the CAR/TCR alone and those which co-express the CAR/TCR in combination with the enhancement module.

This has important safety implications, as it may be undesirable to administer a cell population to a patient which comprises a subpopulation of cells which express the “enhancement module” alone.

Cell

The present invention provides a cell selected by a method according to the second aspect of the invention.

The cell may comprise a nucleic acid sequence, a nucleic acid construct or a vector of the present invention.

The present invention provides a cell engineered to express (a) a nucleic acid sequence which inhibits Fas expression or activity in the cell; and (b) FasL

The cell may or may not also express an NOI as defined above. Where the NOI is co-expressed on the same vector as the nucleic acid sequence which inhibits Fas expression or activity in the cell, this can be used to select for cells expressing the NOI, using the method of the present invention.

However, the invention also has utility where the cell does not co-express and NOI. If a cell, for example a tumour-infiltrating lymphocyte (TIL) is engineered to express express (a) a nucleic acid sequence which inhibits Fas expression or activity in the cell; and (b) FasL it has greater propensity to survive in vivo and enhanced anti-cancer properties.

Tumour-infiltrating lymphocytes are white blood cells that have left the bloodstream and migrated towards a tumour. They include T cells and B cells and are part of the larger category of ‘tumour-infiltrating immune cells’ which consist of both mononuclear and polymorphonuclear immune cells, (i.e., T cells, B cells, natural killer cells, macrophages, neutrophils, dendritic cells, mast cells, eosinophils, basophils, etc.) in variable proportions. In Adoptive T cell transfer therapy, tumour-infiltrating lymphocytes are removed from a patient's tumour, expanded ex vivo, and then given back to the patient to help the immune system kill the cancer cells.

As mentioned above, the immunosuppressive tumour microenvironment upregulates death ligands such as Fas ligand (FasL). FasL induces apoptosis of immune cells that express death receptors for Fas, such as tumour-infiltrating lymphocytes (TILs). The cell of the present invention has an in-built resistance to FasL-mediated apoptosis so an enhanced capacity to survive and persist in the hostile tumour microenvironment.

Cancer cells also express Fas. Upregulated expression of FasL by the cells of the present invention can enhance their anti-cancer effect by FasL-mediated apoptosis of the cancer cells.

For any of the aspect of the invention mentioned above, the cell may be an effector immune cell. The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a tumour infiltrating lymphocyte (TIL) as described above. The cell may be a T-cell expended from a TIL isolated from a subject having cancer.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s), nucleic acid construct(s) or vector(s) of the invention.

The cells may then by purified, for example, selected on the basis of expression of one or more heterologous nucleic acid sequences.

The effector immune cell is capable of recognising and killing a target immune cell. The target immune cell may be a cytolytic immune cell such as a T-cell or NK cell as defined above.

The invention also provides a method for making a cell of the invention which comprises the step of introducing into a cell a nucleic acid sequence, a nucleic acid construct, a vector, or a kit of vectors according to the invention. The cell may be made by transduction. The cell may be screened, post-transduction by a method according to the second aspect of the invention.

Pharmaceutical Composition

The present invention also relates to a pharmaceutical composition containing a plurality of cells according to or selected by the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, as described above.

The present invention also provides a method for treating a disease in a subject, which comprises the following steps:
(i) administering a pharmaceutical composition to a subject, which pharmaceutical composition comprises a plurality of effector immune cells engineered to be resistant to an immunosuppressant; and
(ii) administering the immunosuppressant to the subject.

The effector immune cells may express variant calcineurin engineered to be resistant to one or more calcineurin inhibitors, for example:
calcineurin A comprising mutations T351E and L354A with reference to the shown as SEQ ID No. 71;
calcineurin A comprising mutations V314R and Y341F and with reference to shown as SEQ ID No. 71; or
calcineurin B comprising mutation L124T and K-125-LA-Ins with reference to shown as SEQ ID No. 72.

Step (ii) may involve administering cyclosporin and/or tacrolimus to the cells or to the patient.

The present invention provides a cell of the present invention for use in treating and/or preventing a disease.

The invention also relates to the use of a cell of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

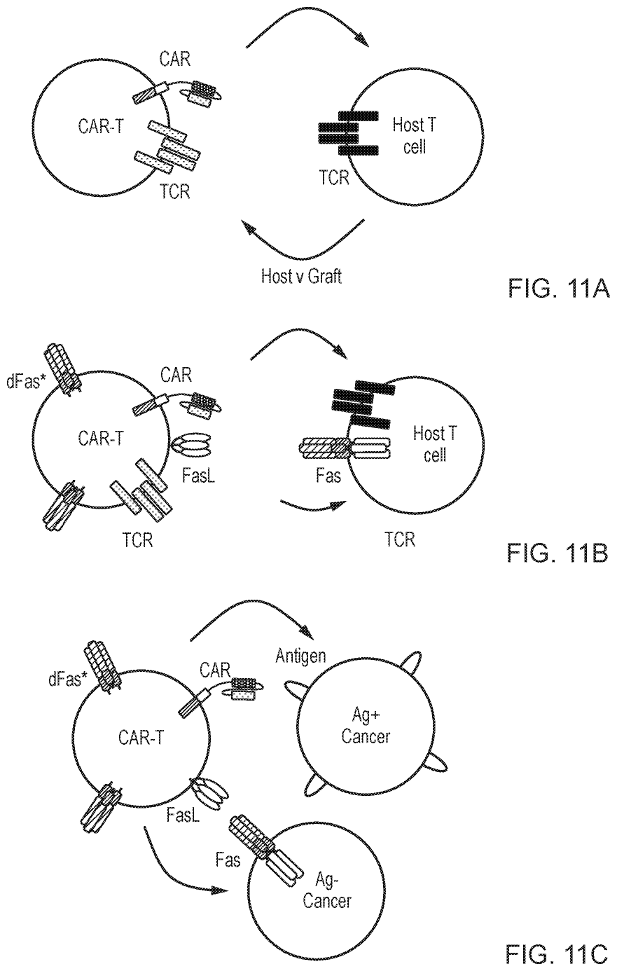

Cells of the present invention which co-express CAR/engineered TCR and FasL are capable of killing both antigen-positive and antigen-negative cells. In this respect they may kill antigen-positive cancer cells via the CAR/TCR; and antigen-negative cancer cells via FasL-mediated apoptosis. This is illustrated schematically in FIG. 11C.

In this respect, the invention provides a method for treating cancer in a subject which comprises cancer cells negative for CAR or TCR target antigen, which comprises the step of administering a plurality of cells according of the invention to the subject. Target-antigen negative cancer cells are killed by FasL binding to Fas on the cancer cells.

Deletion of Alloreactive Cells

In adoptive immunotherapy procedures, there is a risk that T-cells in the host will recognise the administered cells as foreign and reject them, as illustrated schematically in FIG. 11A.

Where the cells of the present invention are modified to a) reduce or eliminate Fas expression and b) express FasL, they will “fight back” and kill the alloreactive host T cells by FasL-mediated apoptosis, as illustrated schematically in FIG. 11B.

Thus, the present invention provides a method for preventing graft rejection in a subject, which comprises the step of administering a plurality FasL-expressing cells of the invention to the subject.

Depletion of alloreactive immune cells can also take place ex vivo or in vitro. In this respect, the invention provides a method for depleting alloreactive immune cells from a population of immune cells, which comprises the step of contacting the population of immune cells with a plurality of FasL-expressing cells of the invention.

Cells intended for transplant can be purified of alloreactive cells by such a method prior to transplant. In this respect, the invention provides an allogeneic or autologous transplant which has been depleted of untransduced cells by such a method.

Selection of Cells Lacking Endogenous TCR

The selection method of the second aspect of the invention enables cells to be selected in which expression of the endogenous TCR has been knocked out, knocked down or in which cell-surface expression of the endogenous TCR has been inhibited.

As the NOI which inhibits expression of endogenous T-cell receptor (TCR) is on the same vector as the nucleic acid sequence which inhibits Fas expression in the cell, cells transduced with this vector will a) lack endogenous TCR expression, and b) be insensitive to FasL. It is therefore possible to select cells, all of which lack endogenous TCR expression, for administration to a subject.

The present invention therefore provides a method for preventing graft versus host disease in a subject, which comprises the step of administering a plurality of cells selected by a method according to the second aspect of the invention, to the subject.

The method may comprise the following steps:
a) transducing a population of cells with a vector co-expressing (i) an NOI which inhibits expression of endogenous TCR and (ii) a nucleic acid sequence which inhibits Fas expression in the cell;
(b) exposing the cells from (a) to FasL; and
(c) administering the cells from (b) to the subject.

Where the cells from (a) also express FasL, step (b) may just involve culturing the cells so that FasL expressed on the cell surface causes elimination of untransduced cells, or cells which do not express the NOI which inhibits expression of endogenous TCR.

The invention will now be further described by way of Examples, which are meant to serve to assist one of ordinary skill in the art in carrying out the invention and are not intended in any way to limit the scope of the invention.

### EXAMPLES

### Example 1—Expression of Truncated Fas Lacking the Death Domain Rescues FasL-Induced Death

Peripheral blood mononuclear cells (PBMCs) were either left untransduced or transduced with one of the constructs shown in the following table:

| Construct: | Expressing: |
|---|---|
| Fmc63-41BBz | CAR alone |
| Fmc63-41BBz-2A-FasL | CAR and Fas ligand |
| Fmc63-41BBz-2A-FasΔDD | CAR and truncated Fas, lacking |
| | the death domain |
| Fmc63-41BBz-2A-FasΔDD-2A-FasL | CAR, truncated Fas, lacking the |
| | death domain, and Fas ligand |

Fmc63-41BBz was a second generation anti-CD19 CAR having an antigen-binding domain derived from Fmc63 and a 41BB-CD3z compound endodomain.

All constructs also expressed the sort-suicide gene, RQR8, which is described in WO2013/153391.

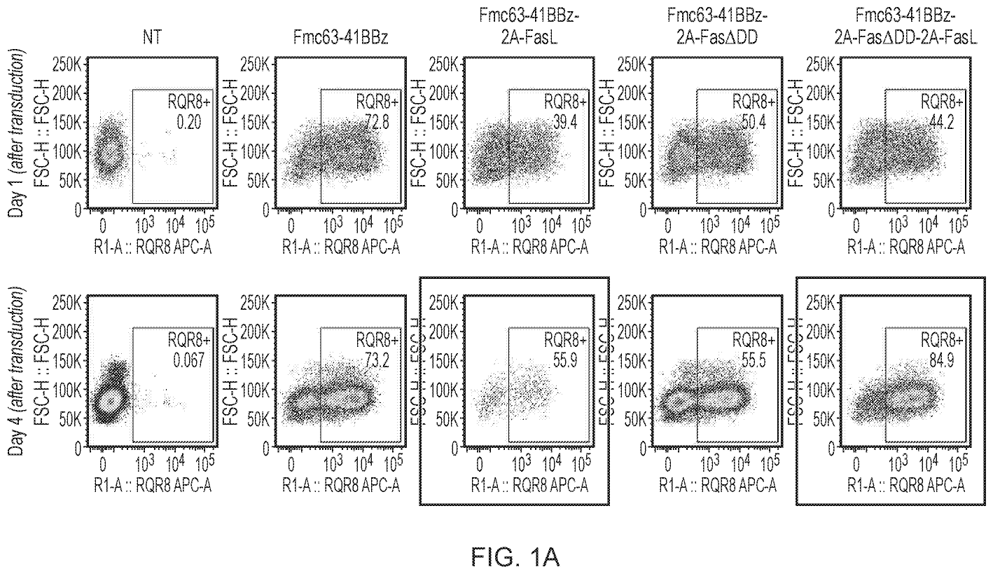

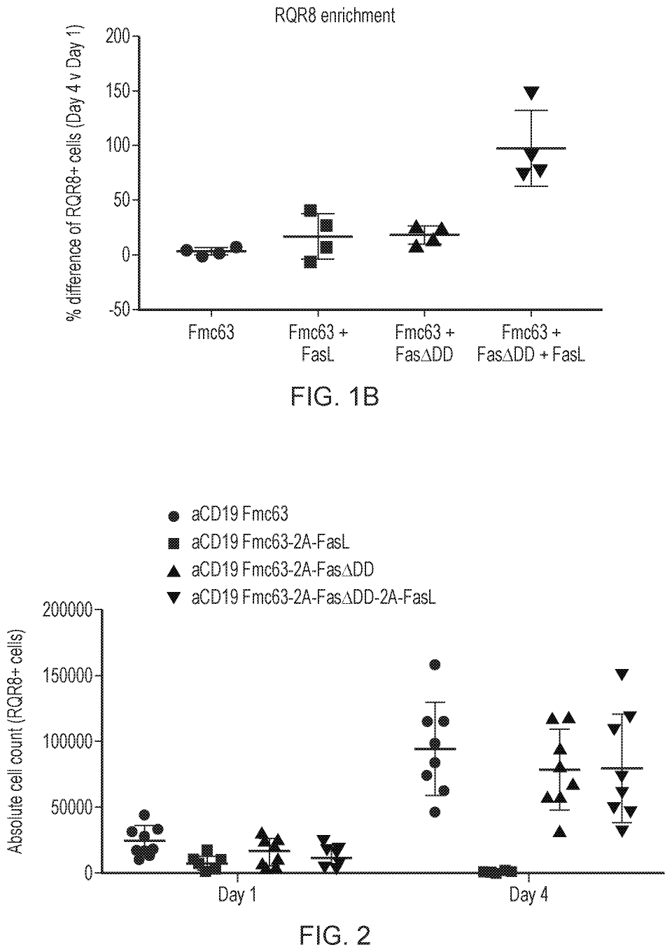

The percentage of cells expressing the constructs were analysed by flow cytometry, one and four days after transduction, by staining for the independent marker, RQR8 and the results are shown in FIG. 1A. The percentage difference of RQR8-expressing PBMCs four days after transduction versus one day after transduction was also calculated and the results are shown in FIG. 1B.

In the cell population expressing FasL, the number of cells (both transduced and untransduced) was dramatically reduced by day 4 (see red box in FIG. 1A), whereas in the cell population co-expressing FasL and a truncated Fas, lacking the death domain, transduced cells survived and were selectively expanding by day 4.

This dominant negative Fas therefore rescues transduced cells from Fas-induced cell death.

In order to investigate proliferation of the cells, in a separate assay using the constructs described above, the day after transduction (day 1) equal numbers of cells were seeded into wells of a 96-well plate and cultured in the presence of IL-2. Absolute cell counts of transduced cells were calculated at day 1 and day 4 by flow cytometry analysis using counting beads, based on expression of RQR8. The results are shown in FIG. 2. Expression of FasL by the cells completely obliterated detectable cells after 4 days of culture (red squares), but co-expression of FasL with dominant negative Fas (FasΔDD) lead to equivalent numbers of cells to those expressing the CAR and FasΔDD without FasL (purple and green triangles). Expression of the dominant negative Fas therefore rescues transduced cells from Fas-induced cell death.

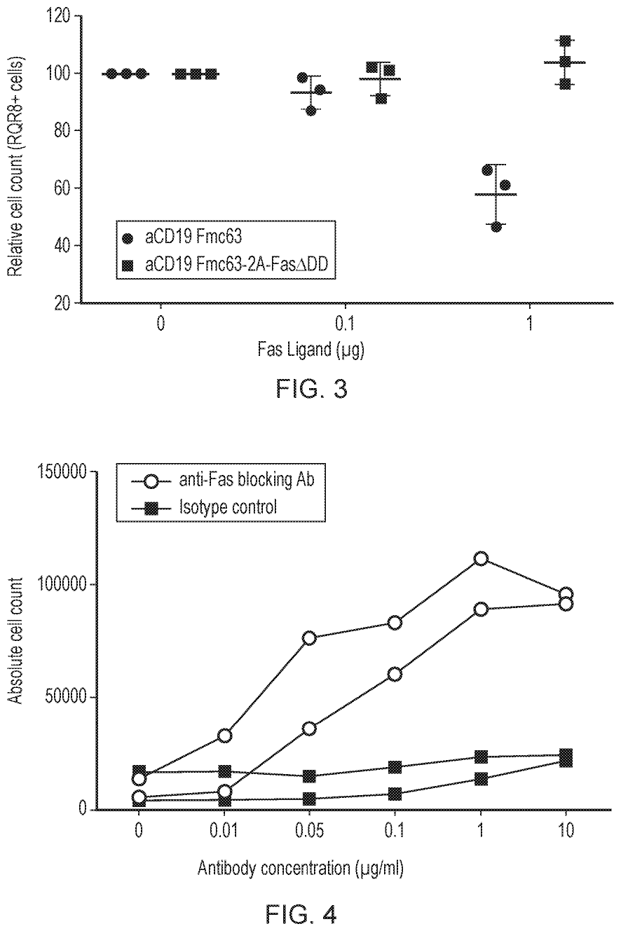

A similar effect was observed using recombinant FasL, as opposed to FasL co-expressed on the PBMCs. Various concentrations of recombinant FasL were immobilised into wells of a 96-well cell culture plate and the following day, PBMCs expressing either the anti-CD19 (fmc63) CAR (circles) or co-expressing the CAR with truncated Fas (FasΔDD, squares) were seeded into wells and cultured for five days. Absolute cell counts of transduced cells were analysed by flow cytometry using counting beads, based on expression of RQR8. The results are shown in FIG. 3. For cells expressing the CAR alone, cell numbers diminished with increasing concentrations of FasL, however this effect was not observed in cells co-expressing the CAR with dominant negative Fas, again showing that the expression of FasΔDD protects the cells from Fas-induced cell death.

In order to demonstrate that the observed effect is due to the interaction between Fas and FasL, PBMCs transduced to co-express the CAR and FasL were treated with either an anti-Fas blocking antibody or an isotype control at various concentrations at the point of transduction. Absolute viable cell counts of PBMCs were calculated three days after transduction and the results from two separate donors are shown in FIG. 4. Viability of the FasL expressing cells increased with increasing concentrations of the anti-Fas blocking antibody, whereas presence of the isotype control showed no effect.

### Example 2—Purification of Transduced Cells Using Hexameric FasL

Peripheral blood mononuclear cells (PBMCs) were either left untransduced or transduced with one of the constructs shown in the following table:

| Construct: | Expressing: |
|---|---|
| RQR8-2A-Fmc63-CD3z | RQR8 and CAR only |
| RQR8-2A-Fmc63-CD3z-2A-Fas-CD40 | RQR8, CAR and a fusion |
| | protein with Fas ectodomain |
| | and CD40 endodomain |
| RQR8-2A-Fmc63-CD3z-2A-Fas-XEDAR | RQR8, CAR and a fusion |
| | protein with Fas ectodomain |
| | and XEDAR endodomain |
| RQR8-2A-Fmc63-CD3z-2A-FasΔDD | RQR8, CAR, and truncated |
| | Fas, lacking the death |
| | domain |

Fmc63-CD3z was a first generation anti-CD19 CAR having an antigen-binding domain derived from Fmc63.

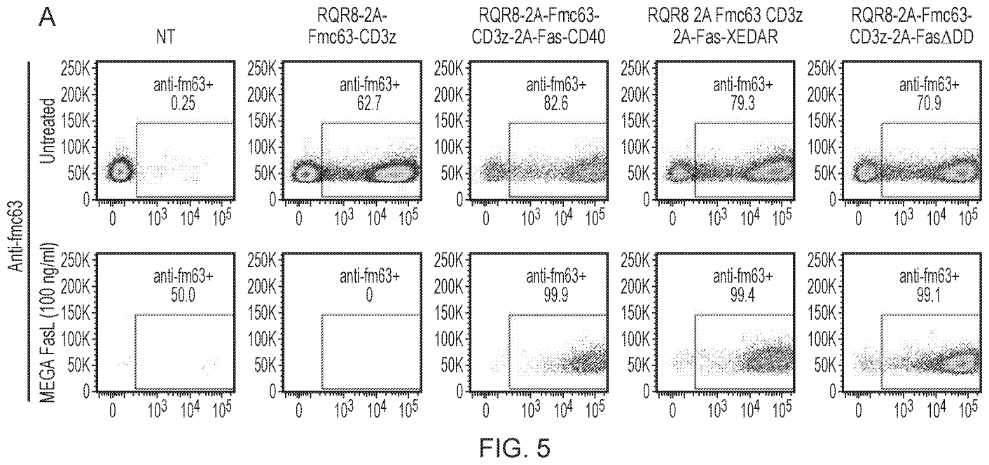

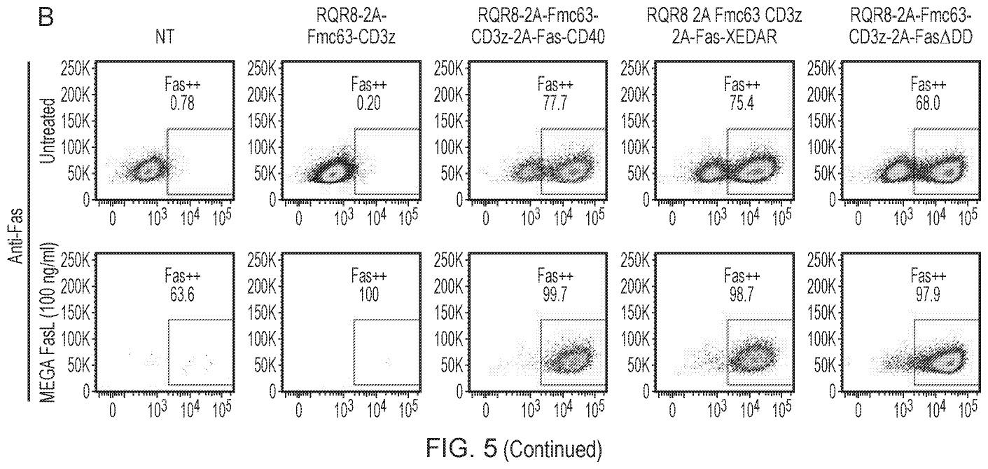

Equal numbers of PBMCs were seeded into wells of a plate in the presence or absence of multimeric FasL and then incubated for three days, at which point cells were analysed by flow cytometry, staining for the Fmc63-CD3z CAR (FIG. 5A) and Fas (FIG. 5B).

The addition of soluble hexameric FasL completely obliterated non-transduced cells and cells transduced with RQR8 and the CAR alone after 3 days. However, for cells transduced to co-express the CAR with any of the three dominant negative Fas molecules (Fas-CD40, Fas-XEDAR and FasΔDD), the addition of soluble hexameric FasL caused apoptosis of the untransduced cells only, leading to purification and enrichment of the transduced cells.

### Example 3—Co-Expression of FasL and dFas can Enhance Killing of Low-Antigen Expressing and Antigen Negative Cancer Cells

Peripheral blood mononuclear cells (PBMCs) were either left untransduced or transduced with one of the constructs shown in the following table:

| Construct: | Expressing: |
|---|---|
| aCD19 Fmc63 | CAR alone |
| aCD19 Fmc63-2A-FasΔDD | CAR and truncated Fas, lacking |
| | the death domain |
| aCD19 Fmc63-2A-FasΔDD-2A-FasL | CAR, truncated Fas, lacking the |
| | death domain, and Fas ligand |

aCD19 Fmc63 was a second generation anti-CD19 CAR having an antigen-binding domain derived from Fmc63 and a 41BB-CD3z compound endodomain.

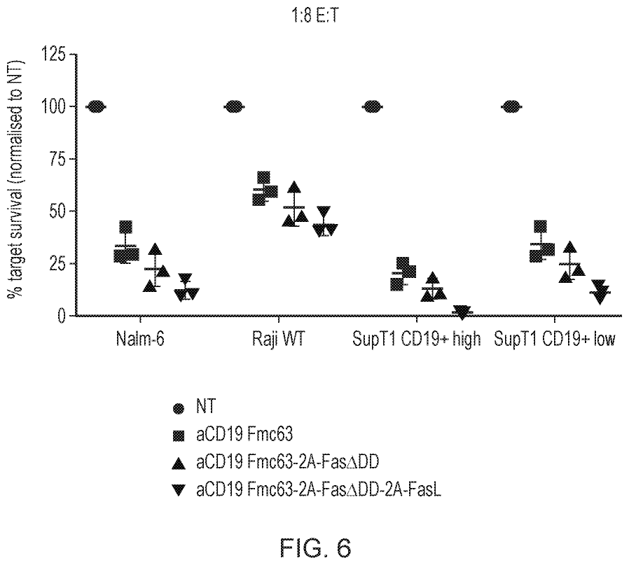

Transduced cells were co-cultured with Nalm-6, Raji, and high and low density CD19-expressing SupT1 cell lines at a 1:8 effector to target ratio for 72 hours. Target cell killing was quantified by flow cytometry and normalised to non-transduced PBMCs and the results are shown in FIG. 6. For all target cells tested, killing was enhanced by the co-expression of dominant negative Fas and enhanced still further by the co-expression of FasL (presumably due to FasL mediated killing of the target cells by the CAR-expressing cells).

These data support the case that FasL-expressing CAR-T cells have two modes of attack against cancer cells: one via the CAR, for antigen-positive target cells; and one via FasL, for antigen negative target cells.

### Example 4—Expression of an Anti-Fas KDEL Molecule Reduces Cell-Surface Expression of Fas

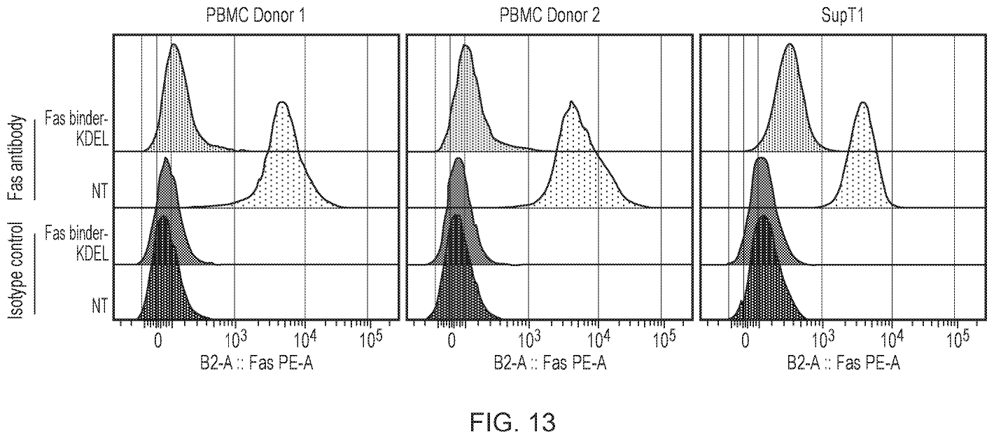

Peripheral blood mononuclear cells (PBMCs) and SupT1 cells were either left untransduced or transduced with a construct expressing the anti-Fas KDEL molecule having the sequence shown as SEQ ID No. 25, together with BFP as a marker for transduction. Three days after transduction the cells were incubated with either a PE-conjugated anti-Fas antibody or a PE-conjugated isotype control and consequently Fas staining was analysed by flow cytometry, with the results shown in FIG. 13. For both PBMCs and SupT1 cells, decreased staining of Fas was observed when transduced with the Fas binder-KDEL polypeptide.

### Example 5—Expression of an Anti-Fas KDEL Molecule Protects a Cell from FasL-Mediated Apoptosis

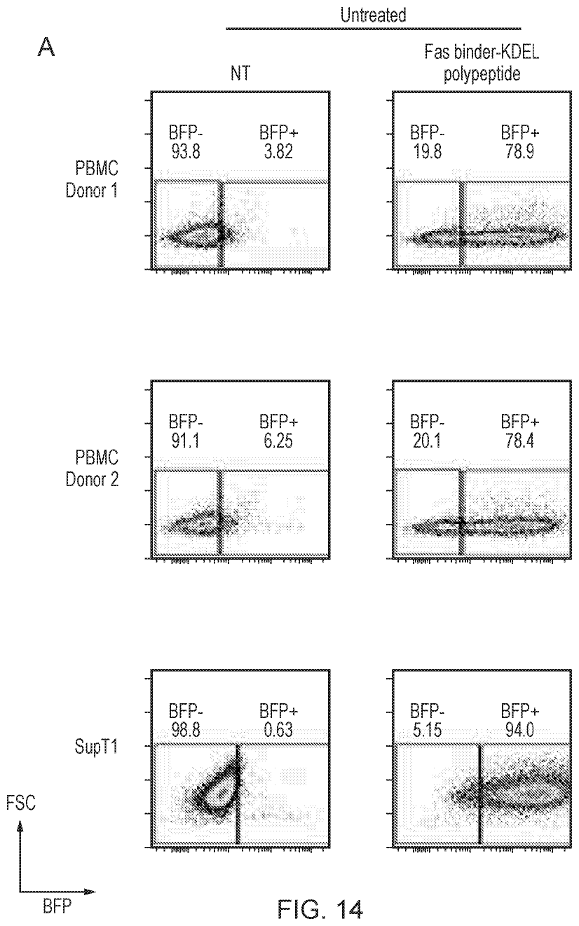

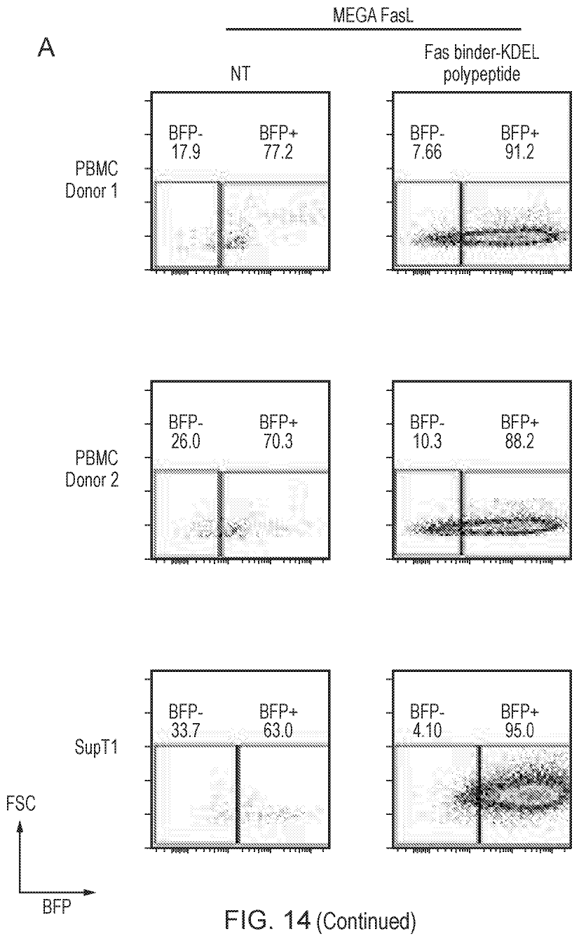

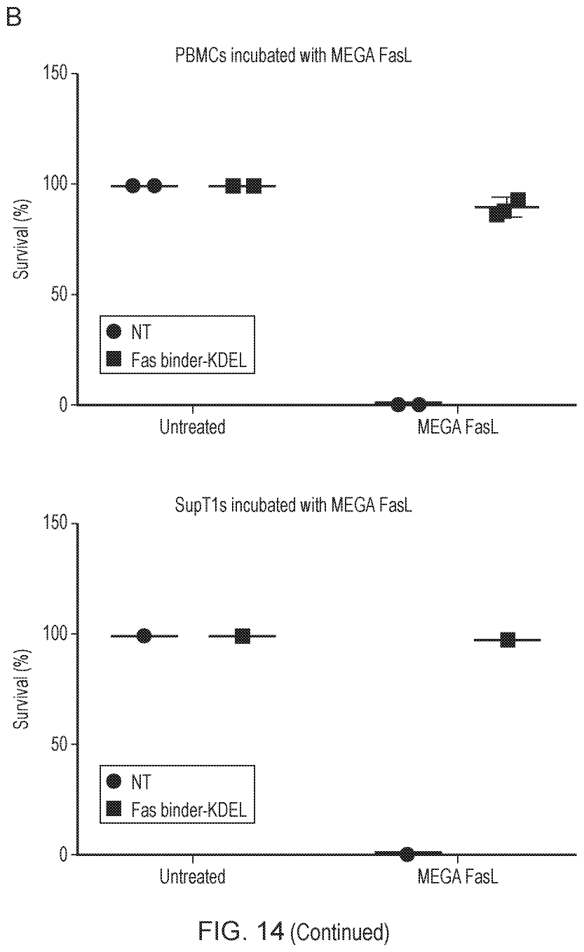

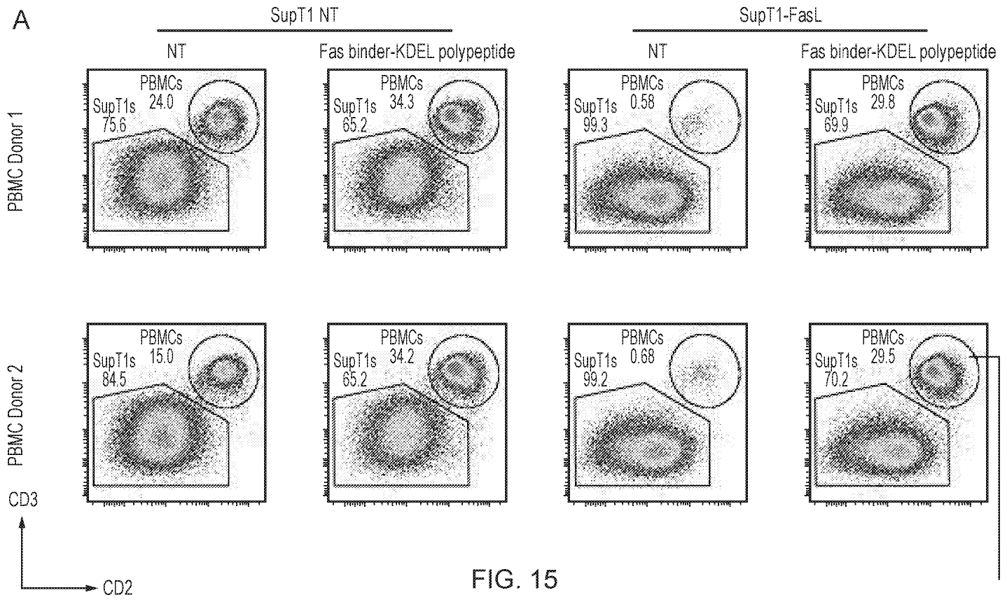

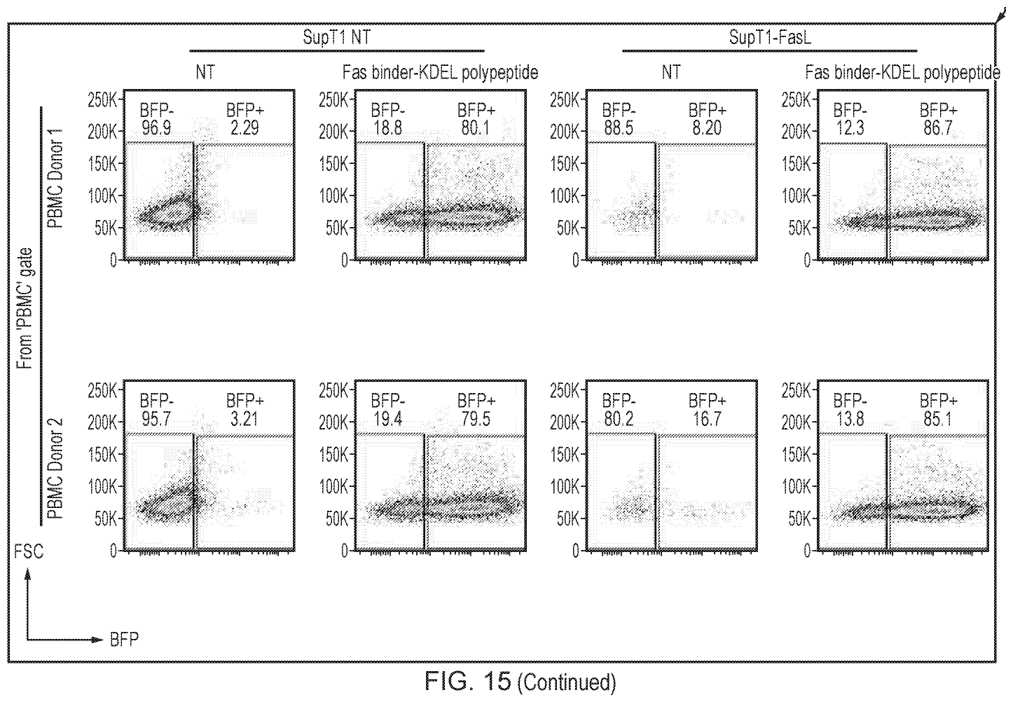

PBMCs and SupT1 cells transduced as described for Example 4 were either left untreated or treated with MEGA FasL (100 ng/mL) for 48 hours, at which point cells were analysed by flow cytometry and the results are shown in FIG. 14. PBMCs and SupT1s transduced with the Fas binder-KDEL polypeptide show almost 100% survival following exposure to MEGA FasL, whereas non-transduced PBMCs and SupT1 cells are completely killed following exposure to the ligand.

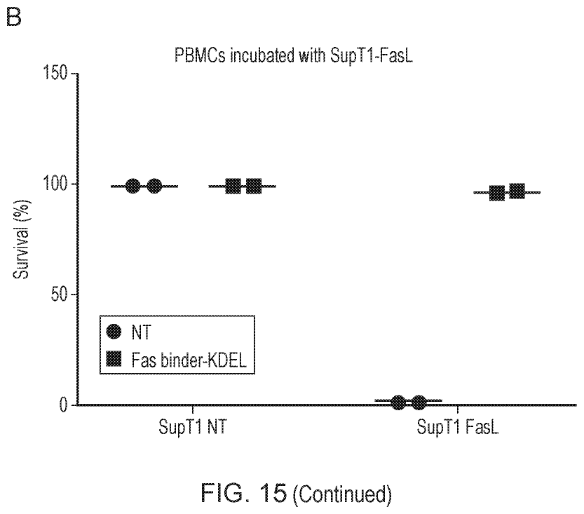

In order to investigate the effect of exposure of the cells to FasL expressed on a cell surface rather than soluble FasL, the experiment above was repeated but this time cells were co-cultured with NT SupT1 cells or FasL-expressing SupT1 cells. Cells were co-cultured at a 1:2 PBMC:SupT1 ratio for 48 hours, at which point cells were analysed by flow cytometry. The results are shown in FIG. 15. Again, PBMCs and SupT1s transduced with the Fas binder-KDEL polypeptide show almost 100% survival following co-culture with FasL-expressing target cells, whereas non-transduced PBMCs and SupT1 cells are virtually undetectable following 48 hours of co-culture.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for selecting for cells transduced to express a nucleic acid sequence of interest (NOI), which comprises the following steps:
(a) transducing a population of cells with a vector co-expressing (i) the NOI and (ii) a nucleic acid sequence which encodes dominant negative Fas and
exposing the cells to FasL;
(b) transducing a population of cells with a vector co-expressing (i) the NOI, (ii) nucleic acid sequence which encodes dominant negative Fas and (iii) a nucleic acid sequence encoding FasL, and culturing the cells; or
(c) co-transducing a population of cells with a first vector co-expressing (i) a CAR or TCR, and (ii) dominant negative Fas, and with a second vector expressing FasL, and culturing the cells.

2-3. (canceled)

4. A method according to claim 1, wherein the dominant negative Fas comprises the Fas extracellular domain but has a truncated or mutated death domain so that it does not bind FADD.

5. A method according to claim 4, wherein the dominant negative Fas comprises the Fas extracellular domain and an endodomain from a TNF receptor.

6. A method according to claim 5, wherein the dominant negative Fas comprises an endodomain from decoy receptor 2 (DcR2), GITR, CD30, XEDAR, CD40, CD27, HVEM, BCMA, 4-1BB or Fn14.

7-10. (canceled)

11. A method according to claim 1 wherein, in step (b), FasL is soluble FasL, FasL bound to a solid substrate, or FasL expressed on the surface of a cell.

12-21. (canceled)

22. A method according to claim 1, wherein the NOI encodes a chimeric antigen receptor (CAR) or a transgenic T cell receptor (TCR).

23. A method according to claim 1, wherein the NOI inhibits expression of endogenous T-cell receptor (TCR).

24. (canceled)

25. A cell engineered to express dominant negative Fas and (b) FasL

26. (canceled)

27. A cell according to claim 25, which also expresses
(c) a chimeric antigen receptor (CAR) or a transgenic T cell receptor (TCR) and/or a nucleic acid sequence of interest (NOI) which inhibits expression of endogenous T-cell receptor (TCR).

28-33. (canceled)

34. A pharmaceutical composition which comprises plurality of cells selected by a method according to claim 1.

35. A method for treating cancer which comprises the step of administering a pharmaceutical composition according to claim 34 to a subject.

36-42. (canceled)

43. A nucleic acid construct for expression in a cell which comprises:
a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR);
a nucleic acid sequence encoding dominant negative Fas; and optionally
a nucleic acid sequence encoding FasL

44. A vector which comprises a nucleic acid construct according to claim 43.

45. (canceled)

46. A kit of vectors which comprises:
a first vector comprising a nucleic acid sequence encoding a chimeric antigen receptor (CAR) or a transgenic T cell receptor (TCR) and/or a nucleic acid sequence of interest (NOI) which inhibits expression of endogenous T-cell receptor (TCR); and a nucleic acid sequence which encodes dominant negative Fas; and
a second vector comprising a nucleic acid sequence encoding FasL

47. A method for making a cell according to claim 25 which comprises the step of introducing into a cell ex vivo: (a) a nucleic acid sequence which encodes dominant negative Fas; and (b) a nucleic acid sequence encoding FasL.

48. A pharmaceutical composition which comprises plurality of cells according to claim 25.

49. A method for treating cancer which comprises the step of administering a pharmaceutical composition according to claim 48 to a subject.
